SEQUENCE LISTING (6 sequences)

SEQ ID NO: 1 - DNA, 64 bp, Artificial Sequence
  Unknown shRNA cassette
ggauccgagccacatccgtccctacttcaagagagtagggacggatgtggctctttttgaaucc

SEQ ID NO: 2 - DNA, 64 bp, Artificial Sequence
  Unknown shRNA cassette
ggauccacctctctcgaccgacacgttcaagagacgtgtcggtcgagagaggttttttgaaucc

SEQ ID NO: 3 - protein, 211 aa, Homo sapiens
MTPWLGLIVLLGSWSLGDWGAEACTCSPSHPQDAFCNSDIVIRAKVVGKKLVKEGPFGTLVYTIKQMKMYRGFTKMPHVQYIHTEASESLCGLKLEVNKYQYLLTGRVYDGKMYTGLCNFVERWDQLTLSQRKGLNYRYHLGCNCKIKSCYYLPCFVTSKNECLWTDMLSNFGYPGYQSKHYACIRQKGGYCSWYRGWAPPDKSIINATDP

SEQ ID NO: 4 - DNA, 5496 bp, Homo sapiens
ctgtggcttgccccagagctgatccttgtctttgtccacttctcagcgaggatggcacttcagggagcccttcccttactatcgcagagagagcaggccctccccagtcatgtccaacccagaactctgttttgttttcttcatagccctagcatcacagaaaatcaccctgtgcattcatggatgtccacgggggcaagggctttgtgttgcttaacccagcatcctgaaccgtgtttgttgaatgaatacagaaccccgtttgctctgggagagcacagaaaacagtcttctatcatatatcatagccagctgcaaacagcagatggcttcccatatcccagagagtaagaaccagagagagagagaaagagagagagtttgggtctttctcctctgtgcctgctctctccagagaaactggaggggtagcagttagcattcccccgctggttccaccaagcacagtcaaggtctctaggacatggccacccctcacctgtggaagcggtcctgctggggtgggtgggtgttagttggttctggtttgggtcagagacacccagtggcccaggtgggcgtggggccagggcgcagacgagaaggggcacgagggctccgctccgaggacccagcggcaagcaccggtcccgggcgcgccccagcccacccactcgcgtgcccacggcggcattattccctataaggatctgaacgatccgggggcggccccgccccgttaccccttgcccccggccccgccccctttttggagggccgatgaggtaatgcggctctgccattggtctgagggggcgggccccaacagcccgaggcggggtccccgggggcccagcgctatatcactcggccgcccaggcagcggcgcagagcgggcagcaggcaggcggcgggcgctcagacggcttctcctcctcctcttgctcctccagctcctgctccttcgccgggaggccgcccgccgagtcctgcgccagcgccgaggcagcctcgctgcgccccatcccgtcccgccgggcactcggagggcagcgcgccggaggccaaggttgccccgcacggcccggcgggcgagcgagctcgggctgcagcagccccgccggcggcgcgcacggcaactttggagaggcgagcagcagccccggcagcggcggcagcagcggcaatgaccccttggctcgggctcatcgtgctcctgggcagctggagcctgggggactggggcgccgaggcgtgcacatgctcgcccagccacccccaggacgccttctgcaactccgacatcgtgatccgggccaaggtggtggggaagaagctggtaaaggaggggcccttcggcacgctggtctacaccatcaagcagatgaagatgtaccgaggcttcaccaagatgccccatgtgcagtacatccatacggaagcttccgagagtctctgtggccttaagctggaggtcaacaagtaccagtacctgctgacaggtcgcgtctatgatggcaagatgtacacggggctgtgcaacttcgtggagaggtgggaccagctcaccctctcccagcgcaaggggctgaactatcggtatcacctgggttgtaactgcaagatcaagtcctgctactacctgccttgctttgtgacttccaagaacgagtgtctctggaccgacatgctctccaatttcggttaccctggctaccagtccaaacactacgcctgcatccggcagaagggcggctactgcagctggtaccgaggatgggcccccccggataaaagcatcatcaatgccacagacccctgagcgccagaccctgccccacctcacttccctcccttcccgctgagcttcccttggacactaactcttcccagatgatgacaatgaaattagtgcctgttttcttgcaaatttagcacttggaacatttaaagaaaggtctatgctgtcatatggggtttattgggaactatcctcctggccccaccctgccccttctttttggttttgacatcattcatttccacctgggaatttctggtgccatgccagaaagaatgaggaacctgtattcctcttcttcgtgataatataatctctatttttttaggaaaacaaaaatgaaaaactactccatttgaggattgtaattcccacccctcttgcttcttccccacctcaccatctcccagaccctcttccctttgcccttctcctccaatacataaaggacacagacaaggaacttgctgaaaggccaaccatttcaggatcagtcaaaggcagcaagcagatagactcaaggtgtgtgaaagatgttatacaccaggagctgccactgcatgtcccaaccagactgtgtctgtctgtgtctgcatgtaagagtgagggagggaaggaaggaactacaagagagtcggagatgatgcagcacacacacaattccccagcccagtgatgcttgtgttgaccagatgttcctgagtctggagcaagcacccaggccagaataacagagctttcttagttggtgaagacttaaacatctgcctgaggtcaggaggcaatttgcctgccttgtacaaaagctcaggtgaaagactgagatgaatgtctttcctctccctgcctcccaccagacttcctcctggaaaacgctttggtagatttggccaggagctttcttttatgtaaattggataaatacacacaccatacactatccacagatatagccaagtagatttgggtagaggatactatttccagaatagtgtttagctcacctagggggatatgtttgtatacacatttgcatatacccacatggggacataagctaatttttttacaggacacagaattctgttcaatgctgttaaatatgccaatagtttaatctcttctattttgttgtcgttgcttgtttgaagaaaatcatgacattccaagttgacatttttttttcattttaattaaaatttgaaattctgaacaccgtcagcaccctctcttccctatcatgggtcatctgacccctgtccgtctccttgtccctgcttcatgtttgggggcctttctttaactgccttcctggcttagctcagatggcagatgagagtgtagtcaagggcctgggcacaggagggagagctgcagagtgtcctgcctgccttggctggagggacacctctcctgggtgtggagacagcttggttccctttccctagctccctggtgggtgaatgccacctcctgagatcctcacctcttggaattaaaattgttggtcactggggaaagcctgagtttgcaaccagttgtagggtttctgttgtgtttttttttttttttttgaaataaaactataatataaattctcctattaaataaaattattttaagttttagtgtcaaaagtgagatgctgagagtaggtgataatgtatattttacagagtgggggttggcaggatggtgacattgaacatgattgctctctgtctcttttttcagcttatgggtatttatcttctattagtatttgtatcttcagttcattccactttaggaaacagagctgccaattgaaacagaagaagaaaaaaaaaaaaagcagcagacaacacactgtagagtcttgcacacacacaagtgcccaggcaaggtgcttggcagaaccgcagagtgggaagagagtaccggcatcgggtttccttgggatcaatttcattaccgtgtacctttcccattgtggtcatgccatttggcagggggagaatgggaggcttggccttctttgtgaggcagtgtgagcagaagctgatgccagcatgtcactggttttgaagggatgagcccagacttgatgttttgggattgtccttattttaacctcaaggtctcgcatggtggggcccctgaccaacctacacaagttccctcccacaagtggacatcagtgtcttctctgtgaggcatctggccattcgcactccctggtgtggtcagcctctctcacacaaggaggaacttgggtgaaggctgagtgtgaggcacctgaagtttccctgcggagtcgataaattagcagaaccacatccccatctgttaggccttggtgaggaggccctgggcaaagaagggtctttcgcaaagcgatgtcagagggcggttttgagctttctataagctatagctttgtttatttcacccgttcacttactgtataatttaaaatcatttatgtagctgagacacttctgtatttcaatcatatcatgaacattttattttgctaaatcttgtgtcatgtgtaggctgtaatatgtgtacattgtgtttaagagaaaaatgaaacccacatgccgccattttcctgaatcaaattctgcagtggaatggagaggaaaatacttctaggcaagcagctagactggtgaattgggggaaatagaaggaactagtaactgagactcctccagcctcctccctattggaatcccaatggctcctggagtaggaaaaaagtttaaactacattcatgttcttgttctgtgtcactcggccctgggtagtctaccatttacttcaccccaagtcctgctgcccatccagttgggaagccatgattttcctaagaatccagggccatgggagatacaattccaagttctcgcttcctcctttgggcatctcttctgcctcccaatcaaggaagctccatgctcaggctctcagctctcgggccagtgctctgctctgtccagggtaggtaatactgggagactcctgtcttttaccctcccctcgttccagacctgcctcatggtggcaacatggttcttgaacaattaaagaaacaaatgactttttggaatagccctgtctagggcaaactgtggcccccaggagacactacccttccatgccccagacctctgtcttgcatgtgacaattgacaatctggactaccccaagatggcacccaagtgtttggcttctggctacctaaggttaacatgtcactagagtatttttatgagagacaaacattataaaaatctgatggcaaaagcaaaacaaaatggaaagtaggggaggtggatgtgacaacaacttccaaattggctctttggaggcgagaggaaggggagaacttggagaatagtttttgctttgggggtagaggcttcttagattctcccagcatccgcctttccctttagccagtctgctgtcctgaaacccagaagtgatggagagaaaccaacaagagatctcgaaccctgtctagaaggaatgtatttgttgctaaatttcgtagcactgtttacagttttcctccatgttatttatgaattttatattccgtgaatgtatattgtcttgtaatgttgcataatgttcactttttatagtgtgtcctttattctaaacagtaaagtggttttatttctatcacaaaaaaaaaaa

SEQ ID NO: 5 - protein, 352 aa, Homo sapiens
MAPLGYFLLLCSLKQALGSYPIWWSLAVGPQYSSLGSQPILCASIPGLVPKQLRFCRNYVEIMPSVAEGIKIGIQECQHQFRGRRWNCTTVHDSLAIFGPVLDKATRESAFVHAIASAGVAFAVTRSCAEGTAAICGCSSRHQGSPGKGWKWGGCSEDIEFGGMVSREFADARENRPDARSAMNRHNNEAGRQAIASHMHLKCKCHGLSGSCEVKTCWWSQPDFRAIGDFLKDKYDSASEMVVEKHRESRGWVETLRPRYTYFKVPTERDLVYYEASPNFCEPNPETGSFGTRDRTCNVSSHGIDGCDLLCCGRGHNARAERRREKCRCVFHWCCYVSCQECTRVYDVHTCK

SEQ ID NO: 6 - DNA, 2988 bp, Homo sapiens
gcaggagggcccagcgacgccgccgcgccagctcccagggcccggccccccccggcgctcacgctctcggggcggactcccggccctccgcgccctctcgcgcggcgatggccccactcggatacttcttactcctctgcagcctgaagcaggctctgggcagctacccgatctggtggtcgctggctgttgggccacagtattcctccctgggctcgcagcccatcctgtgtgccagcatcccgggcctggtccccaagcagctccgcttctgcaggaactacgtggagatcatgcccagcgtggccgagggcatcaagattggcatccaggagtgccagcaccagttccgcggccgccggtggaactgcaccaccgtccacgacagcctggccatcttcgggcccgtgctggacaaagctaccagggagtcggcctttgtccacgccattgcctcagccggtgtggcctttgcagtgacacgctcatgtgcagaaggcacggccgccatctgtggctgcagcagccgccaccagggctcaccaggcaagggctggaagtggggtggctgtagcgaggacatcgagtttggtgggatggtgtctcgggagttcgccgacgcccgggagaaccggccagatgcccgctcagccatgaaccgccacaacaacgaggctgggcgccaggccatcgccagccacatgcacctcaagtgcaagtgccacgggctgtcgggcagctgcgaggtgaagacatgctggtggtcgcaacccgacttccgcgccatcggtgacttcctcaaggacaagtacgacagcgcctcggagatggtggtggagaagcaccgggagtcccgcggctgggtggagaccctgcggccgcgctacacctacttcaaggtgcccacggagcgcgacctggtctactacgaggcctcgcccaacttctgcgagcccaaccctgagacgggctccttcggcacgcgcgaccgcacctgcaacgtcagctcgcacggcatcgacggctgcgacctgctgtgctgcggccgcggccacaacgcgcgagcggagcggcgccgggagaagtgccgctgcgtgttccactggtgctgctacgtcagctgccaggagtgcacgcgcgtctacgacgtgcacacctgcaagtaggcaccggccgcggctccccctggacggggcgggccctgcctgagggtgggcttttccctgggtggagcaggactcccacctaaacggggcagtactcctccctgggggcgggactcctccctgggggtggggctcctacctgggggcagaactcctacctgaaggcagggctcctccctggagctagtgtctcctctctggtggctgggctgctcctgaatgaggcggagctccaggatggggaggggctctgcgttggcttctccctggggacggggctcccctggacagaggcggggctacagattgggcggggcttctcttgggtgggacagggcttctcctgcgggggcgaggcccctcccagtaagggcgtggctctgggtgggcggggcactaggtaggcttctacctgcaggcggggctcctcctgaaggaggcggggctctaggatggggcacggctctggggtaggctgctccctgagggcggagcgcctccttaggagtggggttttatggtggatgaggcttcttcctggatggggcagagcttctcctgaccagggcaaggccccttccacgggggctgtggctctgggtgggcgtggcctgcataggctccttcctgtgggtggggcttctctgggaccaggctccaatggggcggggcttctctccgcgggtgggactcttccctgggaaccgccctcctgattaaggcgtggcttctgcaggaatcccggctccagagcaggaaattcagcccaccagccacctcatccccaaccccctgtaaggttccatccacccctgcgtcgagctgggaaggttccatgaagcgagtcgggtccccaacccgtgcccctgggatccgagggcccctctccaagcgcctggctttggaatgctccaggcgcgccgacgcctgtgccaccccttcctcagcctggggtttgaccacccacctgaccaggggccctacctggggaaagcctgaagggcctcccagcccccaaccccaagaccaagcttagtcctgggagaggacagggacttcgcagaggcaagcgaccgaggccctcccaaagaggcccgccctgcccgggctcccacaccgtcaggtactcctgccagggaactggcctgctgcgccccaggccccgcccgtctctgctctgctcagctgcgcccccttctttgcagctgcccagcccctcctccctgccctcgggtctccccacctgcactccatccagctacaggagagatagaagcctctcgtcccgtccctccctttcctccgcctgtccacagccccttaagggaaaggtaggaagagaggtccagccccccaggctgcccagagctgctggtctcatttgggggcgttcgggaggtttggggggcatcaaccccccgactgtgctgctcgcgaaggtcccacagccctgagatgggccggcccccttcctggcccctcatggcgggactggagaaatggtccgctttcctggagccaatggcccggcccctcctgactcatccgcctggcccgggaatgaatggggaggccgctgaacccacccggcccatatccctggttgcctcatggccagcgcccctcagcctctgccactgtgaaccggctcccaccctcaaggtgcggggagaagaagcggccaggcggggcgccccaagagcccaaaagagggcacaccgccatcctctgcctcaaattctgcgtttttggttttaatgttatatctgatgctgctatatccactgtccaacggagttagacgaaaaaaaaaaaaaaaaaa

(19) United States
(12) Patent Application Publication
(10) Pub. No.: US 2015/0359847 A1
(43) Pub. Date: Dec. 17, 2015

(54) METHOD AND COMPOSITION FOR CONFERRING NEUROPROTECTION

(71) Applicants: BLOOD SYSTEMS RESEARCH INSTITUTE, Scottsdale, AZ (US); BOARD OF REGENTS OF THE UNIVERSITY OF TEXAS SYSTEM, Austin, TX (US)

(72) Inventors: Shibani Pati, San Francisco, CA (US); Dash Pramod, Houston, TX (US)

(21) Appl. No.: 14/688,899

(22) Filed: Apr. 16, 2015

Related U.S. Application Data
(60) Provisional application No. 61/980,439, filed on Apr. 16, 2014.
(60) Provisional application No. 61/980,477, filed on Apr. 16, 2014.

(51) Int. Cl. A61K 38/17; A61K 9/00; A61K 38/57

(52) U.S. Cl. CPC A61K 38/1709; A61K 38/57; A61K 9/0019

(57) ABSTRACT

The present invention relates to compositions and methods for neuroprotection. In particular, provided herein are compositions (for example, mesenchymal stem cell secretory factors) for alleviating and/or protecting against neuronal and neural stem cell damage (for example, resulting from traumatic brain injury), and methods of use thereof.

### CROSS-REFERENCE TO RELATED APPLICATIONS

This application claims benefit of priority under 35 U.S.C. §119(e) of U.S. Ser. No. 61/980,439, filed Apr. 16, 2014, and U.S. Ser. No. 61/980,477, filed Apr. 16, 2014, the entire contents of which are incorporated herein by reference in their entireties.

### STATEMENT OF GOVERNMENT RIGHTS

This invention was made in part with Government support under National Institutes of Health grant K18HL102256-01. The Government has certain rights to the invention.

### BACKGROUND

1. Field of Invention

The present invention relates to therapeutic methods and compositions, and more specifically to compositions including neuroprotective polypeptides for alleviating and protecting against neuronal damage and methods of use thereof.

2. Background Information

Traumatic Brain Injury (TBI) is the number one cause of death and disability in the United States between the ages of 1-44 with approximately 53,000 deaths annually. However deaths are only a fraction of the actual number of cases. According to the CDC, there are approximately 1.7 million TBIs per year. In addition to US civilian cases, recent conflicts in Iraq and Afghanistan have resulted in an additional 230,000+TBIs for military personnel since 2000. Patients that survive TBI are faced with chronic post-injury symptoms such as motor and sensory deficits, impaired cognitive capability and neuropsychological symptoms such as anxiety and depression. Furthermore, there is a substantial increased risk of developing epilepsy.

Aside from the initial gross structural damage, TBI pathology is greatly exacerbated by secondary effects that continue months to years later. On a cellular level, the primary injury results in damage to the blood brain barrier (BBB) and to neurons and their axonal and dendritic connections. Traumatic axonal injury, the loss of connection between neurons causes dysfunction in neural circuitry and is thought to contribute to the long-lasting neurological deficits of TBI. The secondary wave of injury is dominated by the initiation of inflammatory responses by resident and infiltrating immune cells. Continued disruption of the BBB allows for entry of blood-born immune cells into the brain and the subsequent release of inflammatory mediators, additionally activation of resident microglia can contribute to injury.

The hippocampus is a key structure in the brain known to be involved in learning and memory. Damage to the human hippocampus results in significant cognitive deficits and is also known to occur following TBI. The hilar neurons of the dentate gyrus are especially vulnerable to TBI. Loss of these interneurons disrupts hippocampal circuitry and causes neurocognitive deficits. Moreover, experimental studies have shown that doublecortin (DCX) positive newborn neurons in the dentate gyrus die soon after TBI, which may also contribute to TBI-induced cognitive deficits. Furthermore, loss of inhibitory neurons in the hilus of the dentate gyrus could potentially decrease the threshold for post-TBI epilepsy, a condition that develops in some patients. Unfortunately, effective treatments to reduce these deleterious consequences of TBI are currently not available.

There are currently no therapeutics drugs or biologics in use that specifically address neuroprotection after TBI or other forms of neuronal injury. As such, there exists a need for therapeutic compositions which provide neuroprotection.

### SUMMARY

The present disclosure is based on the seminal discovery that tissue inhibitor of matrix metalloproteinase-3 (TIMP3) and wingless-type MMTV integration site family, member 3A (Wnt3a) are neuroprotective polypeptides that are useful in alleviating and protecting the neuronal damage, neural stem cell loss and inflammation that ensues post-injury.

Accordingly, in one aspect, the present invention provides a method of protecting against neuronal or neural stem cell death. The method includes administering to a subject having or at risk of having loss of neural function a neuroprotective effective amount of TIMP3, Wnt3a or combination thereof, thereby protecting against neuronal cell death in the subject. In embodiments, the neuronal cell is a neural stem cell.

In another aspect, the present invention provides a method of enhancing neurogenesis and/or improving neurocognitive dysfunction. The method includes administering to a subject in need thereof a neurogenesis effective amount of TIMP3, Wnt3a or combination thereof, thereby enhancing neurogenesis and/or improving neurocognitive dysfunction in the subject.

In yet another aspect, the invention provides a method of treating a neurological disorder in a subject in need thereof. The method includes administering an effective amount of TIMP3, Wnt3a or combination thereof, thereby treating the neurological disorder in the subject.

In yet another aspect, the invention provides pharmaceutical compositions useful for carrying out the methods described herein. In one embodiment, the pharmaceutical composition includes at least two neuroprotective polypeptides, including TIMP3 and Wnt3a; and a pharmaceutical carrier.

In another embodiment, the pharmaceutical composition includes one or more nucleic acid molecules encoding at least two neuroprotective polypeptides, including TIMP3 and Wnt3a; and a pharmaceutical carrier.

In yet another aspect, the invention provides a method of reducing or inhibiting inflammation of the central nervous system, i.e., the brain in a subject in need thereof. The method includes administering an effective amount of TIMP3, thereby reducing or inhibiting inflammation in the subject.

### BRIEF DESCRIPTION OF THE DRAWINGS

FIGS. 1A-1E are graphical representations depicting various data relating to intravenous TIMP3 treatment which abrogates hippocampal-dependent neurocognitive decline post-TBI. FIG. 1A is a schematic showing experimental concept. FIG. 1B is a representation depicting data. FIG. 1C is a representation depicting data. FIG. 1D is a representation depicting data. FIG. 1E is a representation depicting data.

FIGS. 2A-2C are graphical representations depicting various data relating to intravenous TIMP3 which preserves vulnerable neuronal populations in the hippocampus. FIG. 2A is a pictorial representation depicting data. FIG. 2B is a representation depicting data. FIG. 2C is a representation depicting data.

FIGS. 3A-3H are graphical representations depicting various data relating to TIMP3 activation of Akt-mTORC1 signaling in neurons. FIG. 3A is a representation depicting data. FIG. 3B is a representation depicting data. FIG. 3C is a representation depicting data. FIG. 3D is a representation depicting data. FIG. 3E is a representation depicting data. FIG. 3F is a schematic representation depicting experimental concept. FIG. 3G is a representation depicting data. FIG. 3H is a representation depicting data.

FIGS. 4A-4E are graphical representations depicting various data relating to intravenous TIMP3 activation of the Akt-mTORC1 pathway in the hippocampus in vivo. FIG. 4A is a representation depicting data. FIG. 4B is a representation depicting data. FIG. 4C is a representation depicting data. FIG. 4D is a representation depicting data. FIG. 4E is a representation depicting data.

FIGS. 5A-5H are graphical representations depicting various data relating to TIMP3 protection of neurons and promotion of neurite outgrowth in vitro. FIG. 5A is a representation depicting data. FIG. 5B is a representation depicting data. FIG. 5C is a representation depicting data. FIG. 5D is a representation depicting data. FIG. 5E is a schematic representation depicting experimental design. FIG. 5F is a representation depicting data. FIG. 5G is a representation depicting data. FIG. 5H is a representation depicting data.

FIGS. 6A-6B are graphical representations depicting various data relating to intravenous TIMP3 preservation of neuronal projections in vivo in the molecular layer of the dentate gyrus following TBI. FIG. 6A is a representation depicting data. FIG. 6B is a representation depicting data.

FIGS. 7A-7B are graphical representations depicting various data relating to pharmacological inhibitors differentially subverting the protective effects of intravenously administered TIMP3. FIG. 7A is a schematic representation depicting experimental concept. FIG. 7B is a representation depicting data.

FIG. 8 is a graphical representation depicting an overview of the therapeutic potential of TIMP3 in TBI.

FIGS. 9A-9B are graphical representations depicting various data in embodiment of the invention. FIG. 9A is a representation depicting data. FIG. 9B is a representation depicting data.

FIGS. 10A-10D are graphical representations depicting various data related to hippocampal phospho-S6RP levels 3 days post-TBI. FIG. 10A is a representation depicting data. FIG. 10B is a representation depicting data. FIG. 10C is a representation depicting data. FIG. 10D is a representation depicting data.

FIGS. 11A-11E are graphical representations depicting various data related to embodiments of the invention. FIG. 11A is a representation depicting data. FIG. 11B is a schematic representation depicting plasmid design. FIG. 11C is a representation depicting data (upper, SEQ ID NO: 1; lower, SEQ ID NO: 2). FIG. 11D is a representation depicting data. FIG. 11E is a representation depicting data.

FIGS. 12A-12B are graphical representations depicting various data related to differential effects of Akt-mTOR pathway inhibitors on the protective effects of intravenously administered TIMP3 7 days post-TBI. FIG. 12A is a representation depicting data. FIG. 12B is a representation depicting data.

FIGS. 13A-13C are graphical representations depicting various data related to TIMP3 induced activation of the Akt-mTOR pathway. FIG. 13A is a representation depicting data. FIG. 13B is a representation depicting data. FIG. 13C is a representation depicting data.

FIGS. 14A-14E are graphical representations depicting various data related to intravenous-MSC protection of new neurons from TBI-induced loss and enhanced neurogenesis in the ipsilateral dentate gyms during acute phase post-TBI. FIG. 14A is a representation depicting data. FIG. 14B is a representation depicting data. FIG. 14C is a representation depicting data. FIG. 14D is a representation depicting data. FIG. 14E is a representation depicting data.

FIGS. 15A-15D are graphical representations depicting various data related to dendritic growth and complexity of ipsilateral hippocampal newborn neurons enhancement by IV-MSCs treatment in TBI. FIG. 15A is a representation depicting data. FIG. 15B is a representation depicting data. FIG. 15C is a representation depicting data. FIG. 15D is a representation depicting data.

FIGS. 16A-16E are graphical representations depicting various data related to intravenous-MSC activation of hippocampal Wnt/β-catenin signaling in TBI mice. FIG. 16A is a representation depicting data. FIG. 16B is a representation depicting data. FIG. 16C is a representation depicting data. FIG. 16D is a representation depicting data. FIG. 16E is a representation depicting data.

FIGS. 17A-17D are graphical representations depicting various data related to IV-MSC increase of Wnt3a levels in serum and lungs. FIG. 17A is a representation depicting data. FIG. 17B is a representation depicting data. FIG. 17C is a representation depicting data. FIG. 17D is a representation depicting data.

FIGS. 18A-18B are graphical representations depicting various data related to intravenous-rWnt3a mimicking of the neuroprotective and neurogenic effects of intravenous-MSCs. FIG. 18A is a representation depicting data. FIG. 18B is a representation depicting data.

FIGS. 19A-19E are graphical representations depicting various data related to Wnt3a treatment improving cognitive functions in TBI mice. FIG. 19A is a schematic representation depicting experimental design. FIG. 19B is a representation depicting data. FIG. 19C is a representation depicting data. FIG. 19D is a representation depicting data. FIG. 19E is a representation depicting data.

FIG. 20 is a graphical representation depicting the scheme of treatments for the experiments presented in Example 2.

### DETAILED DESCRIPTION

The present invention provides neuroprotective polypeptides. In particular, the data presented herein illustrate that TIMP3 and Wnt3a are neuroprotective polypeptides useful in protecting against and alleviating neuronal damage.

Before the present compositions and methods are further described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

MSCs have been shown to have potent therapeutic effects in a number of disorders involving neuronal loss including Traumatic Brain Injury (TBI). However, the molecular mechanism(s) underlying these protective effects are largely unknown. Herein it is demonstrated that Tissue Inhibitor of Matrix Metalloproteinase-3 (TIMP3), a soluble protein released by MSCs, is neuroprotective and enhances neuronal survival and neurite outgrowth in vitro. In vivo in a murine model of TBI, intravenous (IV) recombinant TIMP3 enhances dendritic outgrowth and abrogates loss of hippocampal neural progenitors and mature neurons. Intravenous delivery of recombinant TIMP3 reduces anxiety-like behavior post-TBI and also improves hippocampal-dependent neurocognition. Data herein mechanistically demonstrates in vitro and in vivo that TIMP3-mediated neuroprotection is critically dependent on activation of the Akt-mTORC1 pathway.

In addition to TIMP3, the results shown herein further show that Wnt3a also has neuroprotective effects. As Wnt signaling has been implicated in adult neurogenesis, circulating Wnt levels were measured following IV-MSC administration and a significant increase in serum Wnt3a was found, but not Wnt5a. Concurrent with this increase, increased activation of the Wnt/β-catenin signaling pathway was detected in hippocampal neurons. Furthermore, IV recombinant Wnt3a mimicked the neuroprotective and neurogenic effects observed with IV-MSCs and improved neurocognitive function in TBI mice.

Taken together, the results demonstrated herein identify TIMP3 and Wnt3a as therapeutic candidates for use in protecting against neuronal cell death.

Accordingly, in one aspect, the present invention provides a method of protecting against neuronal cell death. The method includes administering to a subject having or at risk of having loss of neural function a neuroprotective effective amount of TIMP3, Wnt3a or combination thereof, thereby protecting against neuronal cell death in the subject.

In another aspect, the present invention provides a method of enhancing neurogenesis. The method includes administering to a subject in need thereof a neurogenesis effective amount of TIMP3, Wnt3a or combination thereof, thereby enhancing neurogenesis in the subject.

In yet another aspect, the invention provides a method of treating a neurological disorder in a subject in need thereof. The method includes administering an effective amount of TIMP3, Wnt3a or combination thereof, thereby treating the neurological disorder in the subject.

In another aspect, the invention provides pharmaceutical compositions useful in the methods described herein.

As used herein, a “pharmaceutical formulation,” “pharmaceutical carrier,” “pharmaceutical diluent,” and “pharmaceutical excipient” is a formulation containing a neuroprotective polypeptide, such as TIMP3 and/or Wnt3a, or a nucleic acid encoding such a polypeptide; or a formulation containing a combination of a neuroprotective polypeptide or nucleic acid encoding such a polypeptide and a carrier, diluent, excipient, or salt which is compatible with other ingredients of the formulation, and not deleterious to the recipient thereof.

The term “pharmaceutically acceptable salt(s)”, as used herein, unless otherwise indicated, includes salts of acidic or basic groups which may be present in the polypeptides and nucleic acids described herein. The polypeptides and nucleic acids described herein are capable of forming a wide variety of salts with various inorganic and organic acids.

The terms “treat” or “treating” mean prohibiting, alleviating, ameliorating, halting, restraining, slowing or reversing the progression, or reducing the severity of a pathological symptom related to or resultant from neuronal damage. As such, these methods include both medical therapeutic (acute) and/or prophylactic (prevention) administration as appropriate.

The term “a therapeutically effective amount” for treating a particular disease or condition means an amount that is sufficient to ameliorate, or in some manner reduce the symptoms associated with the disease. Such amount may be administered as a single dosage or may be administered according to a regimen, whereby it is effective. The amount may cure the disease but, typically, is administered in order to ameliorate the symptoms of the disease. Repeated administration may be required to achieve the desired amelioration of symptoms.

The term “a neurogenesis effective amount” means an amount that is sufficient to promote growth of a neuron, for example, from a neural stem cell or progenitor cell.

The term “neuroprotective effective amount” means an amount that is sufficient to preserve neuronal structure and/or function.

The term “amelioration” of the symptoms of a particular disorder by administration of a particular pharmaceutical composition refers to any lessening, whether permanent or temporary, lasting or transient that can be attributed to or associated with administration of the composition.

The term “subject” as used herein refers to any individual or patient to which the subject methods are performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

It will be understood that the present invention may utilize varying forms of TIMP3 and Wnt3a. For example, the polypeptides of the present invention may be wild-type proteins, as well as homologs, variants and mutants thereof.

The term “wild-type TIMP-3” refers to the TIMP-3 protein having the amino acid sequence disclosed in Gen Bank Acc. No. NP_{—}000353.1 (human; (SEQ ID NO: 3) and encoded by Gen Bank Acc. No. NM_{—}000362.4 (SEQ ID NO: 4).

The term “wild-type Wnt3a” refers to the Wnt3a protein having the amino acid sequence disclosed in Gen Bank Acc. No. NP_{—}149122.1 (human; (SEQ ID NO: 5) and encoded by Gen Bank Acc. No. NM_{—}033131.3 (SEQ ID NO: 6).

As used herein, a polypeptide “variant” or “derivative” refers to a polypeptide that is a mutagenized form of a polypeptide or one produced through recombination but that still retains one or more desired activities.

The terms “TIMP-3 variant” and “Wnt3a variant” refer to a wild-type protein whose amino acid sequence is altered by one or more amino acids, such as by mutation, substitution or truncation. The variant may have conservative changes, wherein a substituted amino acid has similar structural or chemical properties, e.g., replacement of leucine with isoleucine. The variant may have nonconservative changes, e.g., replacement of a glycine with a tryptophan. Analogous minor variations may also include amino acid deletions or insertions, or both. Guidance in determining which amino acid residues may be substituted, inserted, or deleted may be found using computer programs well known in the art, for example, DNASTAR software (DNASTAR Inc., Madison, Wis.).

“Isolated” or “purified” as those terms are used to refer to preparations made from biological cells or hosts means any cell extract containing the indicated DNA or protein including a crude extract of the DNA or protein of interest. For example, in the case of a protein, a purified preparation can be obtained following an individual technique or a series of preparative or biochemical techniques and the DNA or protein of interest can be present at various degrees of purity in these preparations. Particularly for proteins, the procedures may include for example, but are not limited to, ammonium sulfate fractionation, gel filtration, ion exchange change chromatography, affinity chromatography, density gradient centrifugation, electrofocusing, chromatofocusing, and electrophoresis.

A preparation of DNA or protein that is “substantially pure” or “isolated” should be understood to mean a preparation free from naturally occurring materials with which such DNA or protein is normally associated in nature. “Essentially pure” should be understood to mean a “highly” purified preparation that contains at least 95% of the DNA or protein of interest.

While the neuroprotective polypeptides of the present disclosure, such as TIMP3 and Wnt3a, may be defined by exact sequence or motif sequences, one skilled in the art would understand that peptides that have similar sequences may have similar functions. Therefore, peptides having substantially the same sequence or having a sequence that is substantially identical or similar to the neuroprotective polypeptides described herein are intended to be encompassed. As used herein, the term “substantially the same sequence” includes a peptide including a sequence that has at least 60+% (meaning sixty percent or more), preferably 70+%, more preferably 80+%, and most preferably 90+%, 95+%, or 98+% sequence identity with the wild-type neuroprotective polypeptides described herein which retains the same functional activity.

The term “conservative substitution” is used in reference to proteins or peptides to reflect amino acid substitutions that do not substantially alter the activity (for example, antimicrobial activity) of the molecule. Typically conservative amino acid substitutions involve substitution of one amino acid for another amino acid with similar chemical properties (for example, charge or hydrophobicity). The following six groups each contain amino acids that are typical conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K) 5) Isoleucine (I), Leucine (L), Methionine (M), Valine (V); and 6) Phenylalanine (F), Tyrosine (Y), and Tryptophan (W).

The term “amino acid” is used in its broadest sense to include naturally occurring amino acids as well as non-naturally occurring amino acids including amino acid analogs. In view of this broad definition, one skilled in the art would know that reference herein to an amino acid includes, for example, naturally occurring proteogenic (L)-amino acids, (D)-amino acids, chemically modified amino acids such as amino acid analogs, naturally occurring non-proteogenic amino acids such as norleucine, and chemically synthesized compounds having properties known in the art to be characteristic of an amino acid. As used herein, the term “proteogenic” indicates that the amino acid can be incorporated into a protein in a cell through a metabolic pathway.

The phrase “substantially identical,” in the context of two polypeptides, refers to two or more sequences or subsequences that have at least 60+%, preferably 80+%, most preferably 90-95+% amino acid residue identity, when compared and aligned for maximum correspondence, as measured using a sequence comparison algorithm or by visual inspection.

As is generally known in the art, optimal alignment of sequences for comparison can be conducted, for example, by the local homology algorithm of Smith & Waterman ((1981) Adv Appl Math 2:482), by the homology alignment algorithm of Needleman & Wunsch ((1970) J Mol Biol 48:443), by the search for similarity method of Pearson & Lipman ((1988)Proc Natl Acad Sci USA 85:2444), by computerized implementations of these algorithms by visual inspection, or other effective methods.

The neuroprotective polypeptides may have modified amino acid sequences or non-naturally occurring termini modifications. Modifications to the peptide sequence can include, for example, additions, deletions or substitutions of amino acids, provided the peptide produced by such modifications retains the same or similar activity of the wild-type. Additionally, the peptides can be present in the formulation with free termini or with amino-protected (such as N-protected) and/or carboxy-protected (such as C-protected) termini. Protecting groups include: (a) aromatic urethane-type protecting groups which include benzyloxycarbonyl, 2-chlorobenzyloxycarbonyl, 9-fluorenylmethyloxycarbonyl, isonicotinyloxycarbonyl and 4-methoxybenzyloxycarbonyl; (b) aliphatic urethane-type protecting groups which include t-butoxycarbonyl, t-amyloxycarbonyl, isopropyloxycarbonyl, 2-(4-biphenyl)-2-propyloxycarbonyl, allyloxycarbonyl and methylsulfonylethoxycarbonyl; (c) cycloalkyl urethane-type protecting groups which include adamantyloxycarbonyl, cyclopentyloxycarbonyl, cyclohexyloxycarbonyl and isobornyloxycarbonyl; (d) acyl protecting groups or sulfonyl protecting groups. Additional protecting groups include benzyloxycarbonyl, t-butoxycarbonyl, acetyl, 2-propylpentanoyl, 4-methylpentanoyl, t-butylacetyl, 3-cyclohexylpropionyl, n-butanesulfonyl, benzylsulfonyl, 4-methylbenzenesulfonyl, 2-naphthalenesulfonyl, 3-naphthalenesulfonyl and 1-camphorsulfonyl.

In various embodiments, the neuroprotective polypeptides may be administered by any suitable means, including topical, parenteral, subcutaneous, intraperitoneal, intrapulmonary, intranasal, intravenous, and/or intralesional administration in order to treat the subject. However, in exemplary embodiments, the peptides are formulated for intravenous administration.

In certain embodiments, a pharmaceutical composition includes a pharmaceutically acceptable diluent, carrier, solubilizer, emulsifier, preservative and/or adjuvant.

In certain embodiments, a pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (Remington's Pharmaceutical Sciences, 18th Edition, A. R. Gennaro, ed., Mack Publishing Company (1990).

In certain embodiments, an effective amount of a pharmaceutical composition comprising a neuroprotective polypeptide depends on the therapeutic context and objectives. One skilled in the art will appreciate that the appropriate dosage levels for treatment will vary depending in part on the molecule delivered, the indication for which the neuroprotective polypeptide is being used, the route of administration, and the size (body weight, body surface or organ size) and/or condition (the age and general health) of the patient. In certain embodiments, the clinician may titer the dosage and modify the route of administration to obtain the optimal therapeutic effect. In certain embodiments, a typical dosage may range from about 0.1 μg/kg to up to about 100 mg/kg or more, depending on the factors mentioned above. In certain embodiments, the dosage may range from 0.1 μg/kg up to about 100 mg/kg; or 1 μg/kg up to about 100 mg/kg; or 5 μg/kg up to about 100 mg/kg.

As used herein, a “neurological disorder or condition” includes a disease or condition that is characterized by neuronal injury, damage or functional decline. In various embodiments, a neurological disease or disorder includes, but is not limited to, cognition impairment or decline or memory impairment, dementia, Alzheimer's disease, schizophrenia, Parkinson's disease, Huntington's disease, Pick's disease, Creutzfeld-Jakob disease, head trauma, traumatic brain injury, stroke, CNS hypoxia, cerebral senility, multiinfarct dementia, dementia, an acute neuronal disease, age-related cognitive decline, cardiovascular disease, insomnia, ischemia, spinal cord injury and aneurysm, psychosis, inflammatory brain diseases, multiple sclerosis, prion disease, motor neuron diseases (MND), spinocerebellar ataxia (SCA), spinal muscular atrophy (SMA), amyotrophic lateral sclerosis (ALS), seizure disorder and epilepsy.

The following examples are provided to further illustrate the embodiments of the present invention, but are not intended to limit the scope of the invention. While they are typical of those that might be used, other procedures, methodologies, or techniques known to those skilled in the art may alternatively be used.

### Example 1

### Neuroprotective Effect of TIMP3

This example demonstrates that IV administered TIMP3 imparts a neuroprotective effect and enhances neurite outgrowth both in vitro and in brain-injured animals in vivo by activating the Akt-mTORC1 signaling pathway. In an established mouse model of controlled cortical impact in vivo neuroprotection is demonstrated to be associated with improved cognitive function. Taken together the data strongly suggests that TIMP3 has translational potential as a neuroprotective agent to mitigate the deleterious effects of TBI, a disease area with few effective therapeutic options.

The following experimental methods and procedures were utilized.

Methods

Recombinant TIMP3 and recombinant IR-TIMP3 were obtained from Amgen Inc. (Thousand Oaks, Calif.). Primary antibodies in alphabetical order:—Anti-phospho Akt (Ser⁴⁷³) rabbit polyclonal (Cat#9271), anti-Akt rabbit polyclonal (Cat#9272), anti-β3-Tubulin mouse monoclonal (Cat#4466), anti-β3-Tubulin rabbit monoclonal (Cat#5568), anti-Doublecortin (DCX) rabbit polyclonal (Cat#4604), anti-GAPDH rabbit monoclonal (Cat#5174) and anti-GFP rabbit monoclonal (Cat#2956), anti-phospho S6 ribosomal protein (Ser^{235/236}) rabbit monoclonal (Cat#4857), anti-S6 ribosomal protein mouse monoclonal antibody (Cat#2317), were purchased from Cell Signaling Technologies (Danvers, Mass.). Secondary antibodies for western blot:—donkey anti-rabbit IRdye 680RD (Cat#926-68073), donkey-anti mouse IRdye 680RD (Cat#926-68072), donkey anti-rabbit IRdye 800CW (Cat#926-32213) and donkey-anti mouse IRdye (800CW) (Cat#926-32212) were purchased from LI-COR (Lincoln, Nebr.). Secondary antibodies for tissue staining:—Alexa Fluor-488 labeled goat anti-rabbit (Cat# A-11008) and Alexa Fluor-568 labeled goat anti-mouse (Cat# A-11004) were purchased from Life technologies (Grand Island, N.Y.). Conjugated antibody for Phosflow:—PE-CF574 conjugated Anti-phospho Akt (Ser⁴⁷³) mouse monoclonal antibody (Cat#562465) was purchased from BD Biosciences (San Jose, Calif.). Rapamycin (Cat#553211), Triciribine (Cat#124038) and GM6001 (Cat# CC1010) were purchased from EMD Millipore (Billerica, Mass.). For cell culture, DMEM (Cat#10566-016) and FBS, (Cat#16140) were purchased from Life Technologies (Grand Island, N.Y.). Retinoic acid (Cat# R2625) was purchased from Sigma (St Louis, Mo.).

Controlled Cortical Impact Mouse Model

The mouse controlled cortical impact (CCI) injury model was used as previously described. In brief, mice were anesthetized with 5% isoflurane and 1:1 O₂:N₂ mixture. Animals were mounted on a stereotaxic frame and were secured by ear bars and an incisor bar. Anesthesia was maintained with 2.0% isoflurane and 1:1 O₂:N₂ mixture. A 5-mm-diameter craniotomy was made midway between bregma and lambda on the right side, with the medial edge of the craniotomy 1 mm lateral to midline. Injury was produced using a magnetic impactor mounted at an angle of 110° from the vertical plane. A single impact at a velocity of 4+/−0.2 m/s was used inflict a moderate to severe injury (1.2-1.3 mm impact depth). After injury, the incision was closed. Sham animals underwent identical surgeries except for impact injury. Core body temperature was monitored using a rectal thermometer and maintained at 36.8° C.-37.2° C. with a heating pad throughout the procedure. For treatments, TIMP3 (60.0 μg/kg) or vehicle control (PBS) administration was performed at 2, 24, and 48 hrs after injury via tail vein injections in a total volume of 125 μl. Animal research was performed with approval of the Institutional Animal Care and Use Committee at ISIS Services LLC (San Carlos, Calif., USA). Mice received humane care according to the criteria outlined by the National Research Council's Institute of Laboratory Animal Resources in the “Guide for the Care and Use of Laboratory Animals”. Neurocognitive testing conducted at UT Houston was in compliance with University of Texas Houston Institutional Animal Care and Use Committee.

Elevated Plus Maze

The elevated plus maze was used to assess anxiety behavior in mice. Mice were given 5 mins to explore the maze that was raised two feet above the floor. Initially mice were placed in the center of the maze facing the open arm that was opposite of the experimenter. The maze was located in a dimly lit room (10-20 lux) that was quiet and did not have any noise that would disturb the animal's ability to explore. Following the completion of the test, the animal was removed and the maze was wiped down using 50% alcohol. The frequency of entries into the open and closed arms was recorded with an overhead camera using tracking software (Ethovision, Noldus Information Technology, Leesbury, Va.).

Morris Water Maze

A modified Morris water maze task was used to assess long-term memory. Mice were trained to find the location of a hidden platform in one day. This was followed by probe at 30 mins following the last trial and another probe 24 hrs later. Mice were given 10 training trials with an inter-trial interval of 15 mins. Each trial was initiated by placing the animal into the water maze at one of four randomly chosen starting positions. The animal was allowed to search for the hidden platform for a period of 1 min, and the time to find the platform recorded. If the animal failed to find the hidden platform on any given trial, the experimenter led it there. At thirty minutes and twenty-four hours following the final training trial, animals were tested in a probe trial in which the platform was removed from the tank and allowed to search for a period of 1 min. Movement within the maze was monitored using a video camera linked to hacking software (Ethovision™, Noldus Information Technology, Leesbury, Va.). Measures of memory, including latency to first platform crossing, and number of crossings were recorded.

Context-Dependent Fear Discrimination

Contextual discrimination was assessed in a manner similar to prior studies conducted by the inventors and others. In brief, mice were pre-exposed (without shock) to two contexts sharing certain features (horizontal grid floor, background noise, animal handling to and from the room) while differing in others (differently spaced grids, scent, cues and floor shape). Mice were given two trials, one in each chamber, each day. Mice were placed in the shock chamber and 178 seconds later, a 2 second, 0.75 mA shock was given. In the safe chamber, animals were free to roam for 3 mins and no shock was given. Mice were exposed to the shock and safe chambers once a day for 2 days. Comparing the time spent freezing in each chamber during training assessed discrimination of the two contexts.

Tissue Sectioning

Following treatments mice were subjected to a intra-cardiac perfusion-fixation procedure under isoflurane anesthesia. In brief, following a small incision into the right ventricle, a blunt needle was placed 5 mm into the heart and clamped in place. The left atrium was cut and 30 mls of ice cold PBS was perfused through the mouse using a syringe pump at a constant rate of 4 ml/min. After PBS the procedure was repeated with 30 ml of 4% ice-cold paraformaldehyde (PFA) in PBS. Upon completion of the perfusion procedure, brains were dissected out and placed in 20 mls of 4% PFA for a further 24 hrs before transferring to a 30% sucrose solution for an additional 48 hrs. Following sucrose infiltration, brains were frozen in Tissue-Tek O.C.T.™ compound (Cat#4583, Sakura Fineteck USA, Inc. Torrance, Calif.) using an isopentane/dry ice bath. To prepare coronal sections for tissue staining 20 μm thick sections were obtained using a Leica CM1850™ cryostat (Leica Microsystems, Buffalo Grove, Ill.). Sections were immediately placed in a Storage Solution (30% Glycerol, 30% Ethylene Glycol, 40% 0.1M Phosphate Buffer pH 7.2) and stored at −20° C. until required.

Staining and Microscopy

The same protocol was adopted for both tissue sections and isolated neuronal cultures. Briefly, sections were permeabilized with antibody staining solution:—2% w/v BSA (Cat# A3059. Sigma, St Louis, Mo.), 0.1% Triton X-100™ (Cat# X100. Sigma, St Louis, Mo.), 2% v/v goats serum (Cat# S-1000, Vector Labs, Burlingame, Calif.) in PBS. Sections were incubated overnight with primary antibodies on a rotating shaker at 4° C. Sections were then washed three times for 3 min with PBS, and then incubated with secondary antibodies for 1 hr. After staining, sections were washed in PBS, briefly air dried, and coverslipped using Prolong™ gold anti-fade reagent with DAPI (Cat# P36931, Life Technologies, Grand Island, N.Y.). Isolated neuronal cultures underwent the same staining protocol with the exception of the last step in which Prolong gold anti-fade reagent with DAPI was added to the wells of the culture plate sufficient to cover the material. Images were obtained using a Leica DMI6000-B™ inverted microscope, Leica DFC36-FX™ digital camera and Leica Application Suite™ (L.A.S.) software, version 3.1.

Tissue Culture

SH-SY5Y cells were obtained from ATCC (Cat# CRL-2266, Manassas, Va.) and were cultured in DMEM and 10% FBS up to passage 10. For differentiation of SH-SY5Y cells into a neuronal phenotype, cells were first plated in DMEEM+10% FBS for 24 hrs before exchanging media in DMEM+1% FBS 10 μM retinoic acid. Differentiation was performed for 3 days before experimentation. Hippocampal neurons were prepared according to the protocol by Beaudoin et al. (Nature Protocols 2012; 7(9):1741-1754) with minor modifications. In brief, after trypsin digestion of hippocampi isolated from P1 mouse pups single cell suspensions was created by trituration and plated onto Biocoat™ poly-D-lysine coated 12-well plates (Cat#354470, BD Biosciences, San Jose, Calif.). 2 pups were used per well and cell suspensions were incubated for 10 minutes per well for a total of 6 wells. The serial incubation allowed for variation in cell densities applicable to different experiments but also contributed to the reduction in non-neuronal cells. Typically wells 1-2 were pooled for western blotting and wells 4-5 were used for imaging following treatments. After the initial plating cells were incubated for 2 hrs before media was completely exchanged for maintenance media that was supplemented with 1 μM of the anti-mitotic cytosine arabinoside in order to further reduce non-neuronal cells. For western blot, cells were cultured for 7 days before experimentation. Half of maintenance media was exchanged on day 2 and day 5. For morphology experiments, treatments were initiated upon the introduction of maintenance media and was renewed 48 hrs later for a further 24 hrs of treatment. Experiments were terminated by fixation for 20 minutes with 4% paraformaldehyde after which cells were stored at 4° C. in PBS prior to staining protocols.

Nucleofection of Hippocampal Neurons

Primary hippocampal neuron suspensions were transfected post-trituration and prior to plating. Neurons were ‘Nucleofected’ using a 4D-Nucleofector™ System (Lonza, Walkersville, Md.) in conjunction with a P3 Primary Cell 4D-Nucleofector® X Kit. In brief, 5×10⁵ cells were nucleofected with 1.5 μg DNA resuspended in 100 μl of P3 solution after which cells were plated and cultured per normal protocol.

Pathscan Antibody™ Arrays

Pathscan Antibody™ array kits were purchased from Cell Signaling Technologies (Danvers, Mass.). PathScan® RTK signaling antibody array kit (Fluorescent Readout, Cat#7949) and PathScan™ Akt signaling antibody array kit (Fluorescent Readout, Cat#9700) were performed as per manufacturer's instructions. Imaging was performed on a LI-COR Odyssey™ infrared scanner and pixel densities measured by in house LI-COR Image™ suite software.

PhosFlow™ Cytometry

After experimentation, SH-SY5Y cells were detached from culture ware via trypsin digestion and rapidly mixed with an equal volume of BD cytofix fixation buffer (Cat#554655. BD Biosciences, San Jose, Calif.). After wash steps with PBS cells were permeabilized with BD phosflow Perm Buffer III (Cat#558050. BD Biosciences, San Jose, Calif.) and incubated on ice for 30 mins. After washing in BD stain buffer (Cat#554657. BD Biosciences, San Jose, Calif.) cells were counted and 1×10⁶ cells were used antibody incubation. Negative IgG compensation control was obtained using ABC control beads (Cat# A10344. Life Technologies, Grand Island, N.Y.). Antibody incubations were conducted for 30 mins at RT in the dark. Following wash steps the volume was adjusted to 500 μl and results obtained using and LSR II flow cytometer and analyzed using Flowjo™ cytometric analytical software version 8.8.7.

Western Blotting

Cell lysates for western blotting were prepared with RIPA buffer (Cat#89900. Thermofisher Scientific, Rockford, Ill.) supplemented with phosphatase inhibitor cocktail 2 (Cat# P5726. Sigma, St Louis, Mo.), phosphatase inhibitor cocktail 3 (Cat# P0044. Sigma, St Louis, Mo.) and complete protease inhibitor (Cat#1862209. Thermofisher Scientific, Rockford, Ill.). Complete homogenization was ensured by brief sonication using a Branson Sonifier 150, (Branson Ultrasonics Corporation, Danbury, Conn.). Protein content was quantified with a Pierce BCA protein assay kit per manufacturer's instructions (Cat#23225. Thermofisher Scientific, Rockford, Ill.).

Lysates was analyzed in conjuncture with the LI-COR Odyssey™ infra-red imaging system. In brief, lysates were resolved on Bis-Tris mini gels and transferred overnight at 4° C. onto Immobilon-FL™ PVDF membranes (Cat# IPFL10100. EMD Millipore, Billerica, Mass.) using the X-Cell™ sure-lock western blot system (Life Technologies, Grand Island, N.Y.). Blocking and antibody incubation steps were performed using Odyssey blocking buffer (Cat#927-40000. LI-COR, Lincoln, Nebr.). Primary antibodies were incubated for 2 hrs at RT. After wash steps in PBS-T membranes were incubated with LI-COR™ IF secondary antibodies for 45 mins at RT. After further wash steps images were obtained using a LI-COR™ Odyssey scanner and quantified using LI-COR™ Image Studio software. Where stripping steps were necessary membranes were incubated for 5 mins with LI-COR™ Newblot PVDF stripping buffer (Cat#928-40032. LI-COR, Lincoln, Nebr.).

Hypoxia and MTT Assay

MTT Cell Viability & Proliferation Assay™ was purchased from ScienCell, (Cat#8028. Carlsbad, Calif.) and performed with modifications. In brief, 2×10⁵ SH-SY5Y cells were plated per well of a 12 well plate and differentiated for 2 days. Pre-treatments were conducted 1 hr prior to initiation of hypoxia. Control cells underwent media exchange. For hypoxia, cultures were placed in a hypoxia incubator chamber (Cat#27310. StemCell technologies, Vancouver, BC) and purged of regular air using nitrogen gas for 5 mins at a flow rate of 20 L/min, after which both inlet and outlet values were closed and the chamber was placed inside a regular tissue culture incubator. The chamber was re-gassed after one hour again for 5 mins at a flow rate of 20 L/min. After the hypoxia period, tissue culture plates were removed from the chamber and placed back into a regular tissue culture incubator for 18 hrs. At the time of assay 50 μl of MTT Solution was added to each well and incubated for 1 hr at 37° C. After incubation, add 250 μl of MTT solubilization buffer was added and 200 1 of each test sample was transferred to a well of a 96 well plate. The absorbance was then read using a Polarstar plate reader (BMG Labtech, Cary, N.C.) at a wavelength at 570 nm. Background subtraction was carried out with values from an average of 4 wells of solubilization buffer alone.

Neurite Assays

Neurite Outgrowth Assay Kit™ 3 μm (Cat# NS220) and AXIS™ Axon Isolation Devices, 150 μm, Plasma Bonded chamber slides (Cat# AX15005PB) were purchased from (EMD Millipore, Billerica, Mass.). For neurite outgrowth assays SH-SY5Y cells were initially differentiated for 24 hrs at a density of 4×10⁵ cells per well of a 6-well plate. Cells were then dissociated using kit supplied dissociation buffer and 3×10⁵ cells were replated onto transwell inserts. Treatments were conducted for 24 hrs before fixation with ice-cold methanol. Assay was conducted per manufacturer's instructions.

For AXIS™ Axon Isolation devices, chambers were first sterilized from one side of the groves to the other under flow gradient with 70% ethanol to avoid trapping air in the groves. Afterwards the slides were completely immersed in 70% ethanol for 5 mins. Upon removing the slide from the ethanol bath, chambers were washed twice with PBS under a flow gradient to flush the chambers, the procedure was then repeated with Poly-D-Lysine. Chambers were then incubated with Poly-D-Lysine overnight at 4° C. Prior to plating cells, chambers were flushed with ‘plating media’ (see tissue culture section). Simultaneous plating of the single cell suspension in both wells on side of the groves enabled equilibrium to be obtained and a sufficient number of cell remained in the chamber adjacent to the groves. Hippocampi from 2 pups were used per chamber. Cells were allowed to attach for 4 hrs before excess cells in the wells were detached using the rubber tip from a sterile 1 ml syringe, plating media removed and ‘maintenance media’ with treatments were added. Flow gradients were maintained throughout the culture steps and media exchanges were performed with this in mind.

Sholl Analysis

Sholl analysis on processed images of isolated hippocampal neurons was conducted using the Fiji derivation of Image J. (Hypertext Transfer Protocol://Fiji.sc.Fiji). In brief, processed TIFF images were grey-scaled and rendered 8-Bit using Adobe Photoshop™ (San Jose, Calif.). Images were then imported into Fiji and neurites highlighted under the segmentation plugin and the simple neurite tracer option with Hessian-based analysis. After neurites had been traced the images were rendered as an analyzed skeleton and Sholl analysis was run on the image from the Analyze menu option.

shRNA Plasmid Constructs

Plasmid vector pENN.AAV.U6.ShRLuc.CMV.eGFP.SV40 was provided by the Penn vector core, University of Pennsylvania, (Philadelphia, Pa.). mTOR shRNA sequence was chosen based on the previous publication by Jaworrski et al (43). Sequence ‘mTOR3071’ was chosen based on complete sequence identity between rat and mouse genes. Scrambled sequence (SCR) was designed using the online scramble design tool by genscript on the World Wide Web at genscript.com/ssl-bin/app/scramble. Cassette sequences were designed by S.L.G. and cloning, amplification and sequence verification was performed by MCLAB (South San Francisco, Calif.).

Real-Time PCR

Cells were lysed using 0.7 mL of QIAzol™ reagent (Qiagen, Valencia Calif.), followed by 15 min centrifugation at 12,000 g at 4° C. Total RNA was extracted from the aqueous layer using the miRNeasy Mini™ Kit (Qiagen, Valencia Calif.) with on-column DNAase treatment (Qiagen RNase-Free DNase Set™). RNA was transcribed into cDNA using the SuperScript VILO™ cDNA Synthesis Kit (Life Technologies, Grand Island, N.Y.). Quantitative real-time PCR measuring mouse-specific mTOR and Beta-actin (ACTB) genes using Taqman™ real time PCR was performed using the ABI ViiA 7 Real-Time PCR System™. Raw cycle threshold (Ct) numbers of amplified mTOR gene products were normalized to the housekeeping gene ACTB to control for cDNA input amounts. mRNA relative copy number was determined using the comparative Ct method.

Animal Drug Administration

For animal injections, the stock solutions of both Triciribine and Rapamycin were diluted immediately before i.p. injections with 0.3 ml PBS containing 5% polyethylene glycol 400 and 5% Tween 80. Drug-treated mice received 3 i.p. injections of 1 mg/kg of either inhibitor 30 minutes prior to IV TIMP3 (Specific injection times were therefore 30 minutes, 23.5 and 71.5 hours post-TBI).

MMP Sensolyte Assays

SensoLyte Fluorimetric MMP-2 Activity Assay Kit™ (Cat#71151) and SensoLyte Fluorimetric MMP-9 Activity Assay Kit™ (Cat#71155) were purchased from AnaSpec (Fremont, Calif.) and performed per manufacturer's instructions.

### DESCRIPTION OF FIGURES

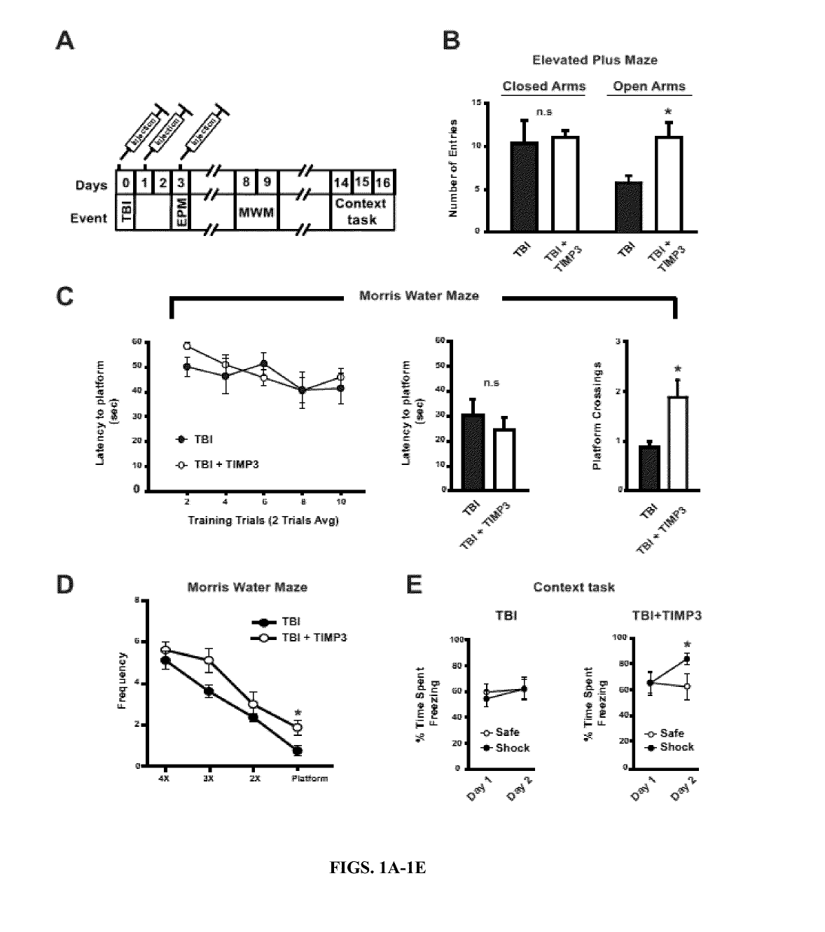

FIGS. 1A-1E are graphical representations depicting various data relating to intravenous TIMP3 treatment which abrogates hippocampal-dependent neurocognitive decline post-TBI. (A) Diagram depicts an overview of the experimental design. TIMP3 administration (60 μg/kg) was performed via tail-vein injection at 1 hr, 24 hrs and 72 hrs post-TBI, TBI alone mice received PBS injections at the same time-points. Elevated Plus Maze (EPM) was performed on day 3 following the last injection, Morris Water Maze over days 8 and 9 and Context-dependent fear discrimination task over days 14-16. (B) Elevated Plus Maze. TIMP3 subjects entered the open arms more frequently than TBI alone subjects (TBI+TIMP3=11±1.8, TBI alone=5.8±0.8; t(15)=2.56, P=<0.05). (C) Morris Water Maze. Left panel, during training the two groups did not significantly differ in the ability to learn the platform location. Middle panel, the two groups did not significantly differ in the latency to first platform crossing. Right panel, TIMP3 treated mice demonstrated an increase in the total number of correct platform crossings (TBI+TIMP3=1.9±0.4; TBI alone=0.9±0.1; t(13)=38.5, P=<0.05). (D) Further analysis of crossing concentric rings surrounding the platform found the TBI+TIMP3 mice spent more time in the area around the platform (F(1,7)=5.73; P=<0.05). (E) Context-dependent fear discrimination. Habituation was conducted on day 14, training was conducted on day 15 (task day 1) and discrimination day was conducted 24 hrs later (task day 2). Left panel, TBI alone mice did not significantly discriminate between the two contexts. Right panel, TBI+TIMP3 mice discriminated between the environments following training (F(1,7)=9.46, P=<0.05).

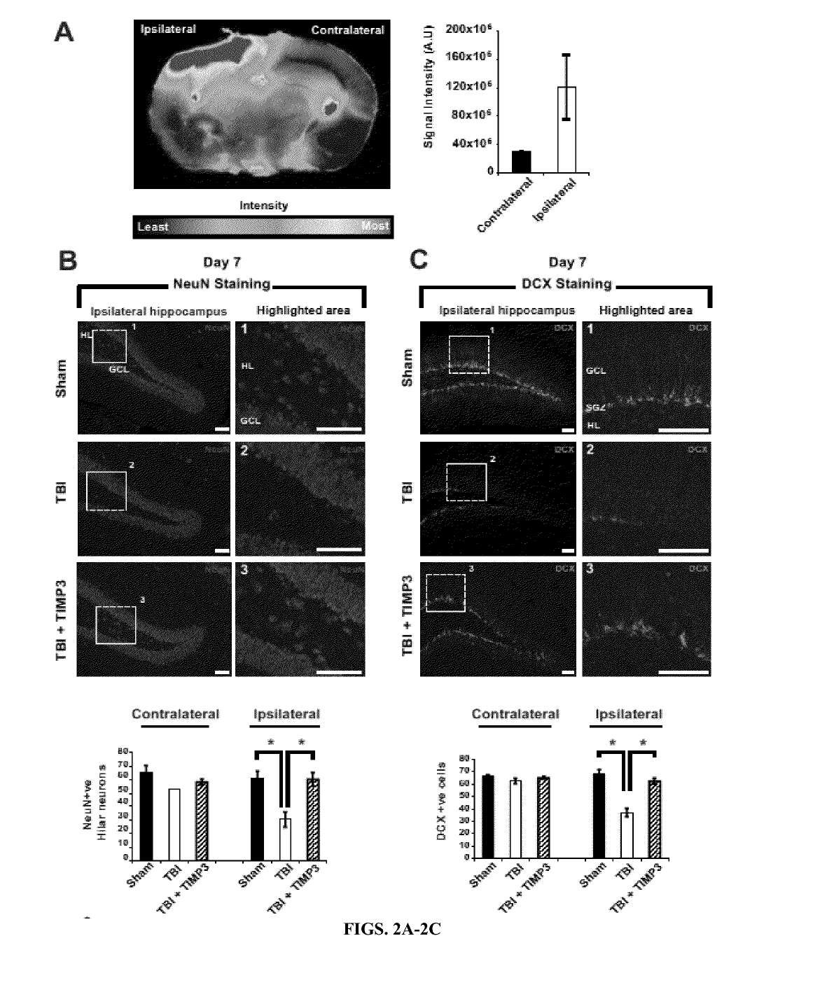

FIGS. 2A-2B are graphical representations depicting various data relating to intravenous TIMP3 which preserves vulnerable neuronal populations in the hippocampus. (A) Coronal section through the impact site showing target coverage following IV administration of an infra-red tagged TIMP3. Spectrum bar indicates the intensity of the distribution. Representative image shows penentrance of TIMP3 into the ipsilateral hippocampus n=2. (B) NeuN staining of post-mitotic mature neurons 7 days post-TBI. Representative photomi-crographs of ipsilateral hippocampi from the 3 groups show NeuN staining in the granule cell layer (GCL) and Hilus (HL) of the dentate gyrus. Highlighted area panels show enhanced view of NeuN+ve hilar interneurons. Scale bar=100 μm. Lower panel, quantification of NeuN+ve neurons in the HL. n=4, 4 section per animal, mean±s.e.m. On the ipsilateral side TBI resulted in a significant reduction in NeuN+ve cells compared to sham controls (Paired t-test, P<0.05). TIMP3 treatment significantly attenuated the loss of NeuN+ve cells (Paired t-test, P<0.05). (C) DCX staining of neural progenitors/immature neurons 7 days post-TBI. Representative photomi-crographs of ipsilateral hippocampi from the 3 groups showing DCX staining in the subgranule zone (SGZ) of the dentate gyrus. Highlighted area panels show enhanced view of DCX positive immature neurons. Scale bar=100 μm. Lower panel, quantification of DCX+ve neurons, n=4, 4 sections per animal, mean±s.e.m. TBI resulted in a significant reduction in ipsilateral DCX+ve cells compared to sham controls (Paired t-test, P<0.05) which was significantly attenuated by TIMP3 treatment (Paired t-test P<0.05).

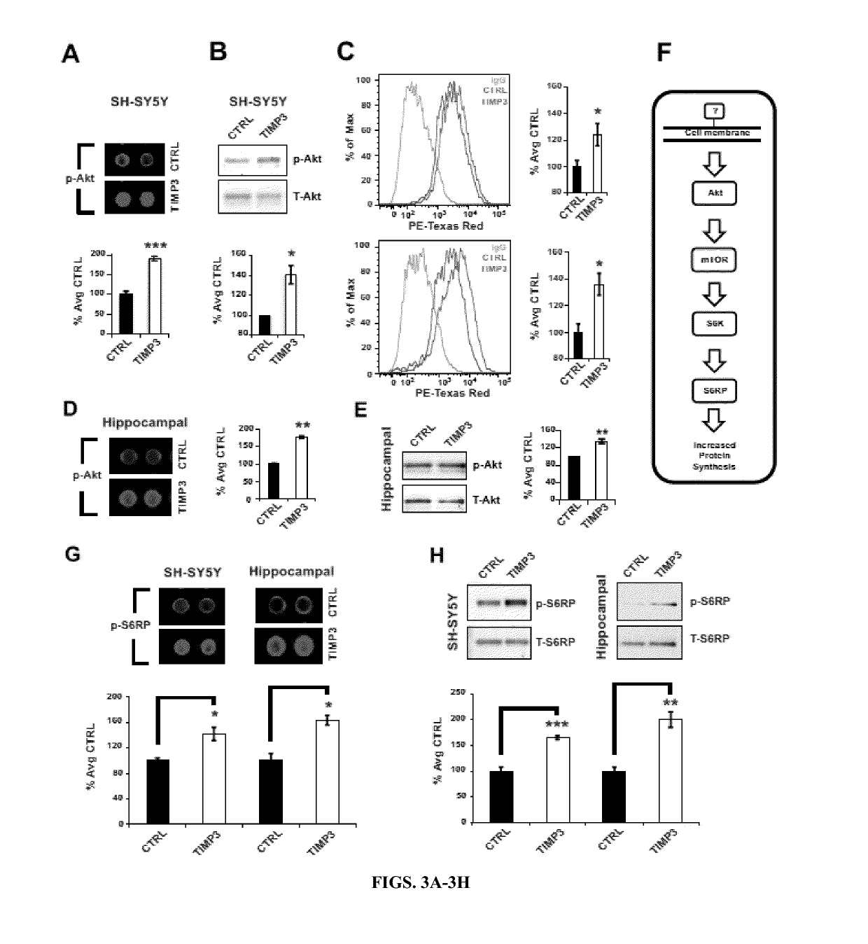

FIGS. 3A-3F are graphical representations depicting various data relating to TIMP3 activation of Akt-mTORC1 signaling in neurons. (A) Fluorescent antibody signaling array. 15 mins treatment with 1 μg/ml TIMP3 activates phospho-AKT(Ser⁴⁷³) in SH-SY5Y cells. (Paired t-test P=<0.001). n=4, mean±s.e.m. (B) Western blot. 15 mins treatment with 1 μg/ml TIMP3 activates p-AKT(Ser⁴⁷³) in SH-SY5Y cells. (Paired t-test P=<0.05). n=4, mean±s.e.m. (C) Phosflow cytometry. 15 mins (Upper panel) and 45 minutes (Lower panel) of treatment with 1 μg/ml TIMP3 activates p-AKT(Ser⁴⁷³) in SH-SY5Y cells. n=4, mean±s.e.m. (Paired t-test, P=<0.05). (D) Fluorescent antibody signaling array. 15 mins treatment with 1 μg/ml TIMP3 activates p-AKT(Ser⁴⁷³) in primary hippocampal neurons. n=4, mean±s.e.m. (Paired t-test, P=<0.01). (E) Western blot. 15 mins treatment with 1 μg/ml TIMP3 activates p-AKT(Ser⁴⁷³) in primary hippocampal neurons. n=4, mean±s.e.m. (Paired t-test, P=<0.01). (F) Signaling diagram indicating the steps of the Akt-mTORC1 pathway between Akt and S6 Ribosomal Protein (S6RP). (G) Fluorescent antibody signaling array shows 15 mins treatment with 1 μg/ml TIMP3 phosphorylates mTORC1 pathway target S6 Ribosomal Protein (S^{235/236}) in SH-SY5Y cells (Left panel, Paired t-test, P=<0.05) and primary hippocampal neurons (Right panel, Paired t-test, P=<0.05). n=4, mean±s.e.m. (H) Western blot confirms 15 mins treatment with 1 μg/ml TIMP3 activates S6 Ribosomal Protein (S^{235/236}) in SH-SY5Y cells (Leftpanel, Paired t-test, P=<0.001) and primary hippocampal neurons (Rightpanel, Paired t-test, P=<0.01). n=4, mean±s.e.m.

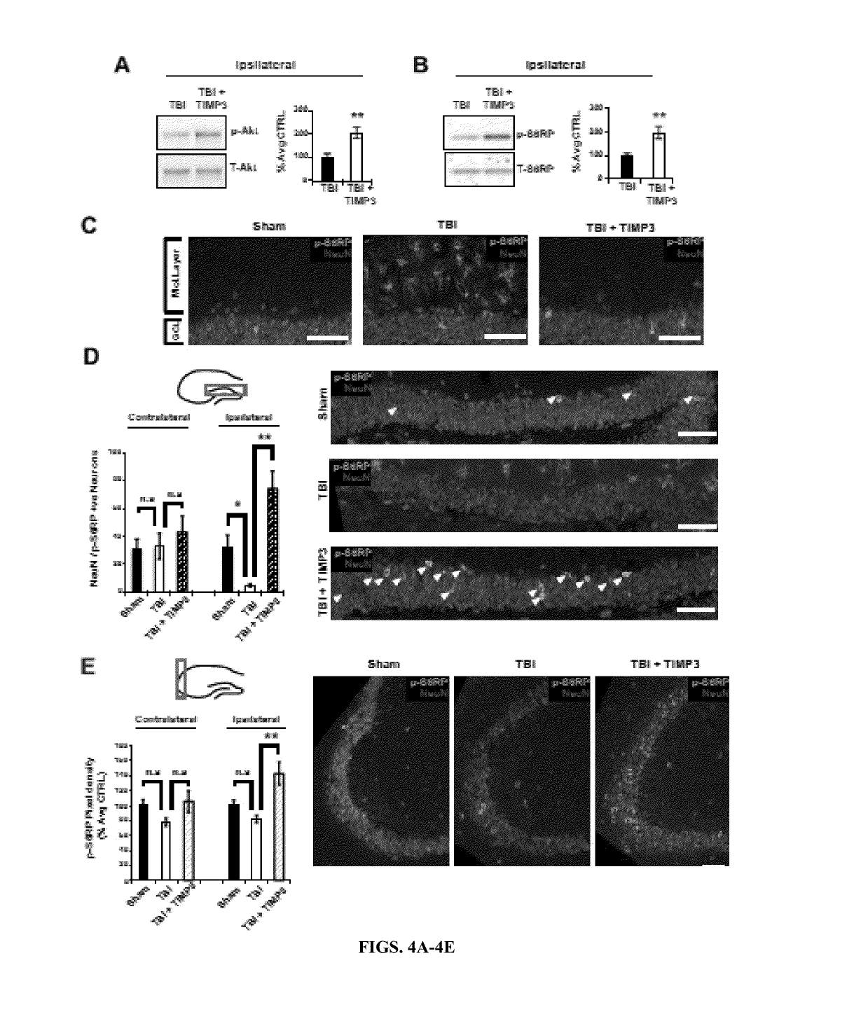

FIGS. 4A-4E are graphical representations depicting various data relating to intravenous TIMP3 activation of the Akt-mTORC1 pathway in the hippocampus in vivo. TIMP3 administration (60 μg/kg) was performed via tail-vein injection at 1 hr, 24 hrs and 72 hrs post-TBI. Animals were sacrificed on day 3, 1 hr after last injection. (A) Western blot. TIMP3 treatment significantly elevates both phospho-Akt (Ser⁴⁷³) and (B) phospho-S6 Ribosomal Protein (S^{235/236}) in ipsilateral hippocampal homogenates. n=8, mean±s.e.m. (Paired t-test, P=<0.01). (C) Representative photomicrographs showing the interface between the granule cell layer (GCL) and the molecular layer (Mol.layer) of the dentate gyrus proximal to the site of injury. Phosphorylated S6RP (S^{235/236}) (green) in the non-neuronal molecular layer was only detected in TBI-injured animals. Scale bar=100 μm. (D) Quantification of phospho-S6RP (S^{235/236}) (green)/NeuN+ve (red) neurons in the GCL proximal to injury site. n=3, 4 sections per animal, mean±s.e.m. Data show a significant reduction with TBI (Paired t-test P<0.05) and an elevation with TIMP3 treatment (Paired t-test P<0.01). Scale bar=100 μm. (E) Quantification of phospho-S6RP (S^{235/236}) (green) pixel density in area CA3. n=3, 4 sections per animal, mean±s.e.m. Data shows a slight reduction with TBI and an significant elevation with TIMP3 treatment. (Paired t-test P<0.05). Scale bar=100 μm.

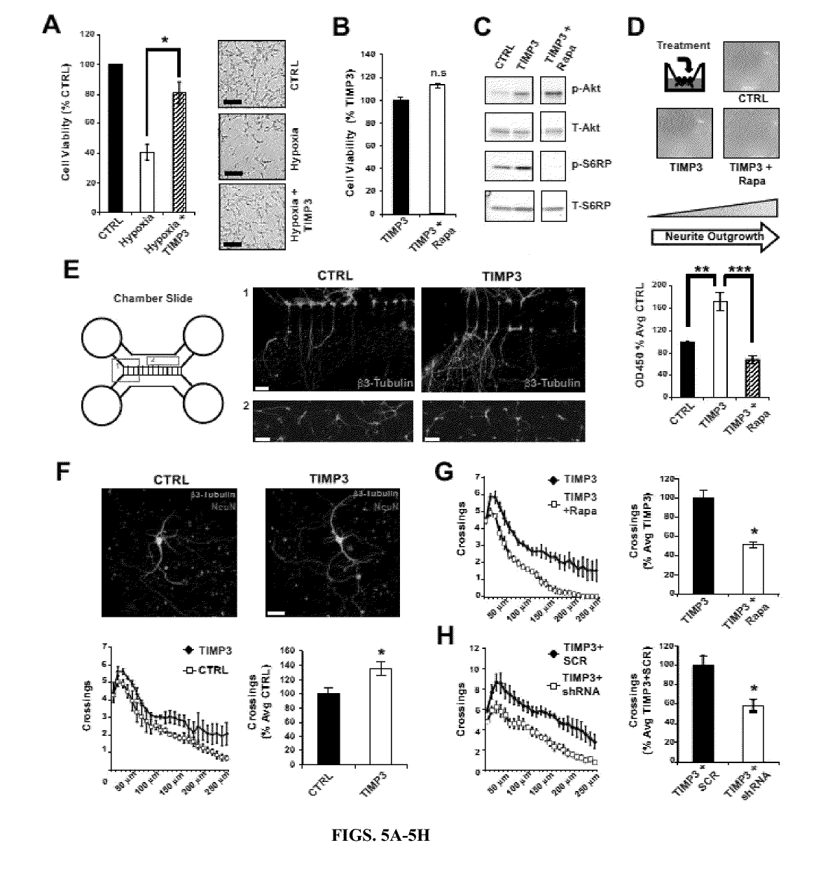

FIGS. 5A-5H are graphical representations depicting various data relating to TIMP3 protection of neurons and promotion of neurite outgrowth in vitro. (A) Hypoxia assay. 5 hrs hypoxia+18 hrs of recovery resulted in a significant reduction in SH-SY5Y cell viability measured by MTT assay. 1 hr pre-treatment with 1 ng/ml TIMP3 prevents loss of cell viability. n=4, mean±s.e.m. (Paired t-test, P=<0.05). Photomicrographs representative of result. Scale bar: 100 μm. (B) MTT Assay. 1 nM of mTORC1 pathway inhibitor rapamycin (Rapa) did not affect viability. n=3, mean±s.e.m. (C) Western blot. Akt (Ser473) phosphorylation was not inhibited with rapamycin. n=4, mean±s.e.m. (D) Colorimetric neurite outgrowth assay. 48 hrs treatment of SH-SY5Y cells with 1 μg/ml TIMP3 promotes increased neurite outgrowth. Cartoon depicts set up of treatment to cell bodies. Photomicrographs indicate colorimetric change due to increased neurite production, representative of result. n=4, mean±s.e.m. (Paired t-test, P=<0.05). (E) Neurite chamber assay. Cartoon depicts orientation of the chamber and location of photomicrographs. Cells bodies are located in one chamber and neurites grow through channels into the opposite chamber. Upper photomicrographs (1) indicate 7 days treatment of hippocampal neurons with TIMP3 results in increased neurite outgrowth. Lower photomicrographs (2) indicate comparable cell densities. Scale bar: 250 μm. (F) Sholl analysis. 72 hrs treatment of hippocampal neurons with 1 μg/ml TIMP3 significantly increased neurite complexity. 20 neurons were analyzed per treatment, per experiment, n=3, mean±s.e.m. (Paired t-test, P=<0.05). Photomicrographs representative of result. Scale bar: 50 μm. (G) Sholl analysis. 72 hrs treatment of hippocampal neurons with 1 nM rapamycin significantly inhibited the effects of TIMP3 on neurite outgrowth. n=3, mean±s.e.m. (Paired t-test, P=<0.05) (H) Sholl analysis. shRNA against mTOR significantly inhibited the effects of TIMP3 on neurite outgrowth versus control scrambled sequence (SCR). Hippocampal neurons were cultured for 7 days post-transfection prior to analysis. n=3, mean±s.e.m. (Paired t-test, P=<0.05).

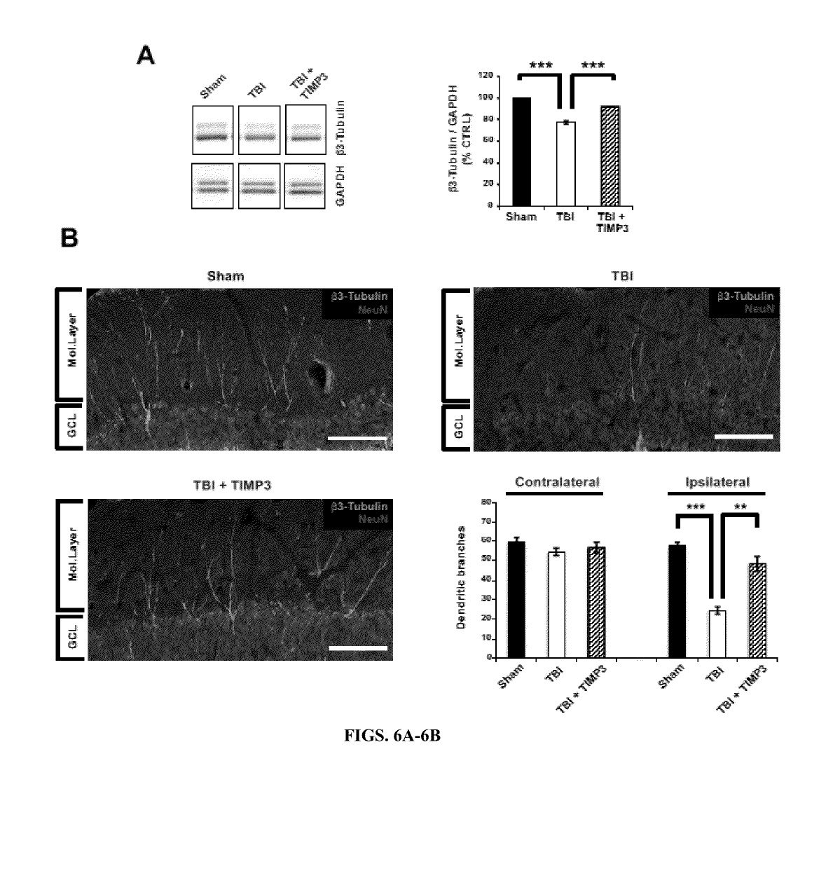

FIGS. 6A-6B are graphical representations depicting various data relating to intravenous TIMP3 preservation of neuronal projections in vivo in the molecular layer of the dentate gyrus following TBI. TIMP3 administration (60 μg/kg) was performed via tail-vein injection at 1 hr, 24 hrs and 72 hrs post-TBI. Animals were sacrificed on day 7. (A) Western blot. Samples from ipsilateral hippocampus show a reduction in neuronal cytoskeletal protein p3-tubulin 7 days post-TBI that is attenuated by TIMP3 treatment. Ubiquitous cytosolic protein GAPDH was used as a loading control. n=4, mean±s.e.m. (Paired t-test, P=<0.001). (B) Representative photomicrographs showing the interface between the granule cell layer (GCL) and the molecular layer (Mol.layer) of the dentate gyrus proximal to the site of injury. Sections were stained for p3-tubulin (green) and NeuN (red) for location. Scale bar=100 μm. Quantification of the number of observable p3-tubulin+ve dendritic branches in the molecular layer greater than 50 μm in length. n=4, 4 section per animal, mean±s.e.m. Data show a reduction with 1BI that was attenuated by TIMP3 treatment. (Paired t-test, **=P<0.01, ***P=<0.001).

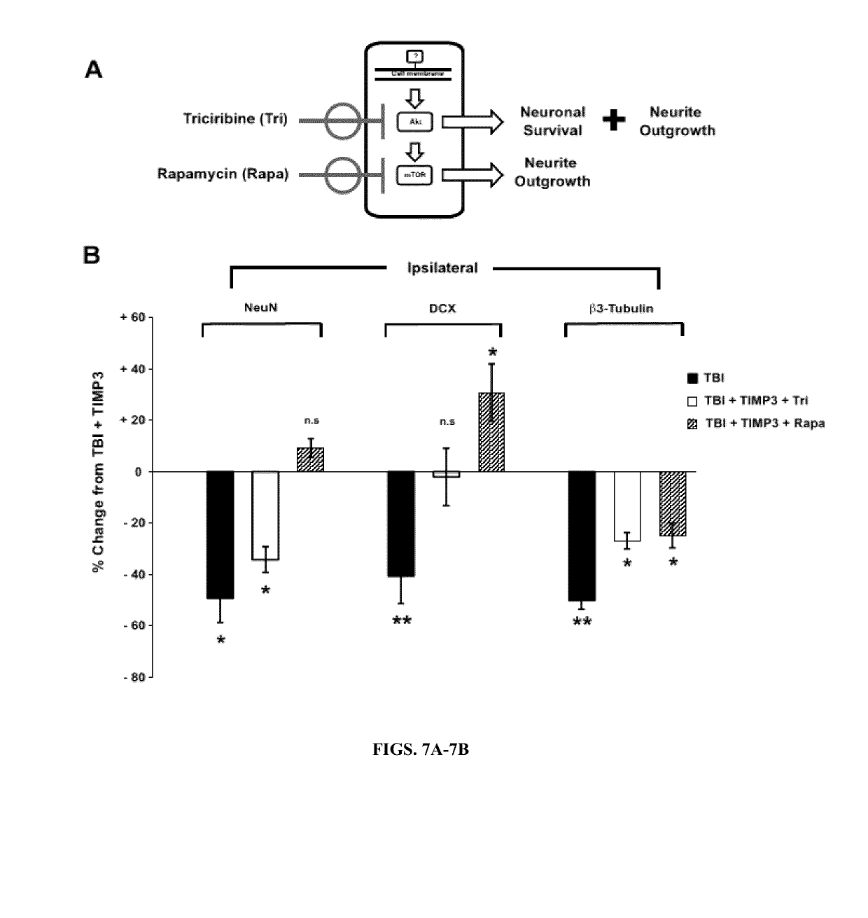

FIGS. 7A-7B are graphical representations depicting various data relating to pharmacological inhibitors differentially subverting the protective effects of IV TIMP3. (A) Diagram depicts an overview of the experimental design and predicted results. (B) Quantification of tissue staining from the Ipsilateral hippocampus 7 days post TBI. Bar chart shows percent change from TIMP3 treatment post-TBI. Triciribine as predicted attenuated both neuronal survival (NeuN) and neurite outgrowth (p3-tubulin). Rapamycin as predicted only attenuated neurite outgrowth. DCX neuronal progenitors demonstrate a mixed reaction to the effects of Triciribine and Rapamycin treatment. For each stain n=4, 4 sections per animal, mean±s.e.m (Paired t-test, *=P<0.05, P=<0.01).

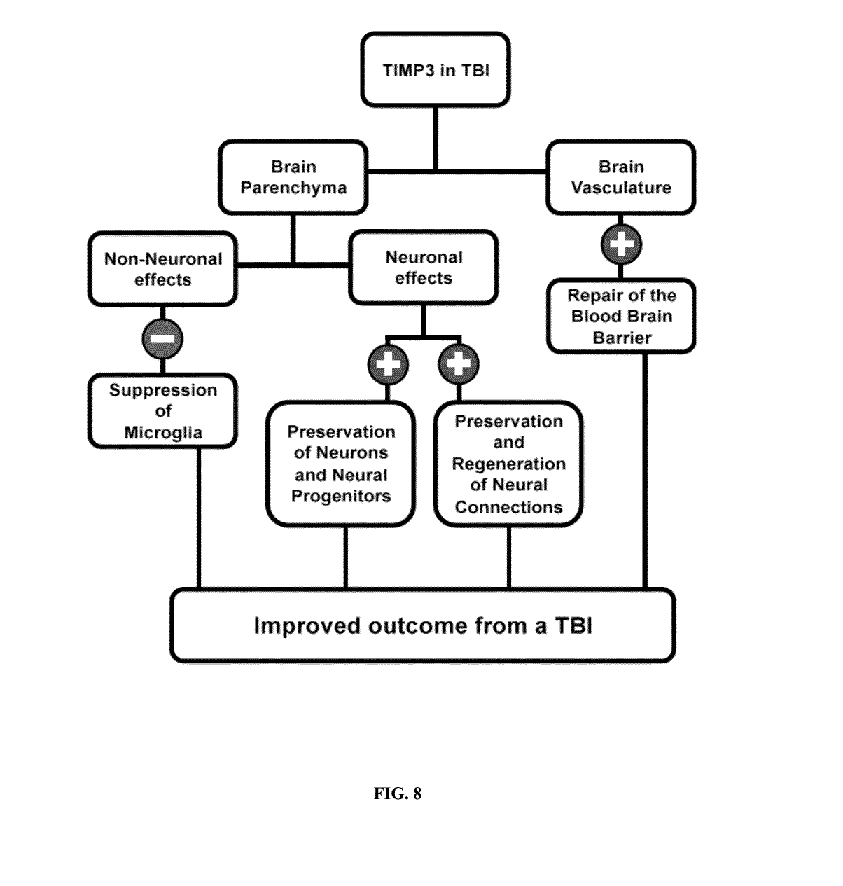

FIG. 8 is a graphical representation depicting an overview of the therapeutic potential of TIMP3 in TBI. Diagram summarizes the pleio-tropic potential of TIMP3 as a therapeutic for TBI. Outside of the brain parenchyma TIMP3 has been shown to reduce BBB compromise, preventing further infiltration of circulating cells. Within the brain TIMP3-initiated intra-neuronal signaling imparts neuroprotection of neuronal populations and either the generation or repair of neuronal connections. Additionally TIMP3 either directly or indirectly prevents microglia activity. Taken together, TIMP3 has multiple effects that correlate with improved neu-rocognition and outcome from a TBI.

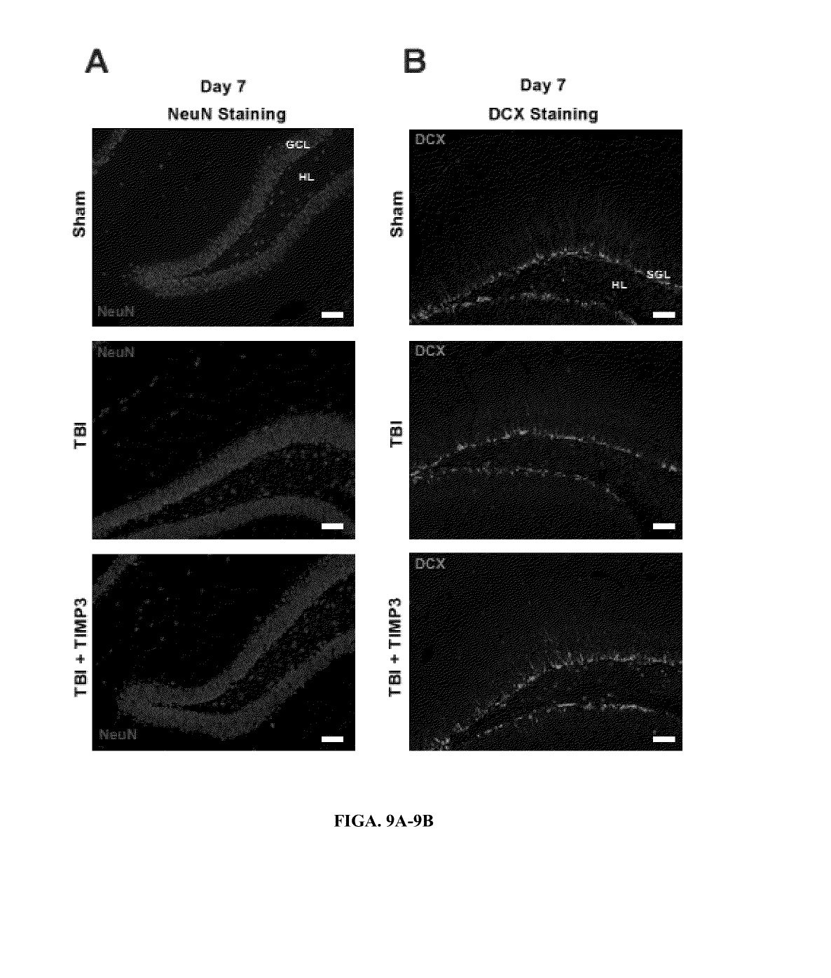

FIGS. 9A-9B are graphical representations depicting various data in embodiment of the invention. No differences were found between groups for NeuN and DCX staining in contralateral hippocampi 7 days post-TBI. (A) NeuN staining of mature-neurons. Representative photomicrographs of contralateral hippocampi from the 3 groups showing no differences in NeuN staining in the granule cell layer (GCL) and Hilus (HL) of the dentate gyrus. Scale bar=100 μm. (B) DCX staining of neural progenitors/immature neurons. Representative photomicrographs from the 3 groups showing no differences in DCX staining in the subgranule cell layer (SGL) of the dentate gyrus from contralateral hippocampi. Scale bar=100 μm.

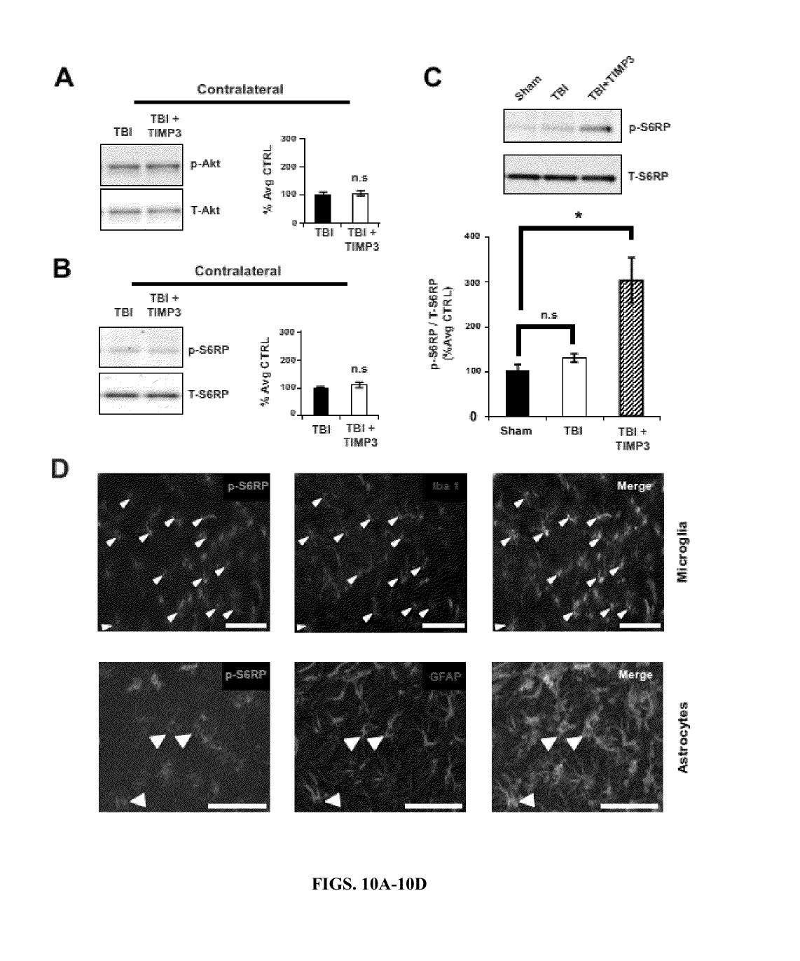

FIGS. 10A-10D are graphical representations depicting various data related to hippocampal phospho-S6RP levels 3 days post-TBI. Animals were sacrificed 1 hr after last injection. (A) Western blot. No differences were observed in contralateral hippocampal homogenates between conditions for Phospho-Akt (Ser⁴⁷³) and (B) phospho-S6RP (S^{235/236}). n=8, mean±s.e.m. (C) Western blot. Ipsilateral hippocampal phospho-S6RP levels 3 days post-TBI are not significantly elevated in TBI alone versus sham controls unlike IV TIMP3 treated animals versus Sham controls (Sham vs. TIMP3, Paired t-test, P=<0.05). n=4, mean±s.e.m. (D) Tissue staining in ipsilateral hippocampi 3 days post-TBI in TBI alone mice. Phospho-S6RP co-localizes with microglia and astrocyte markers in the molecular layer of the hippocampus of TBI alone mice. Upper Panel, phospho-S6RP (S^{235/236}) (green) co-localizes with microglia marker Iba1 (red). Lower Panel, phospho-S6RP (S^{235/236}) (green) co-localizes with astrocyte marker GFAP (red). Scale bar=50 μm.

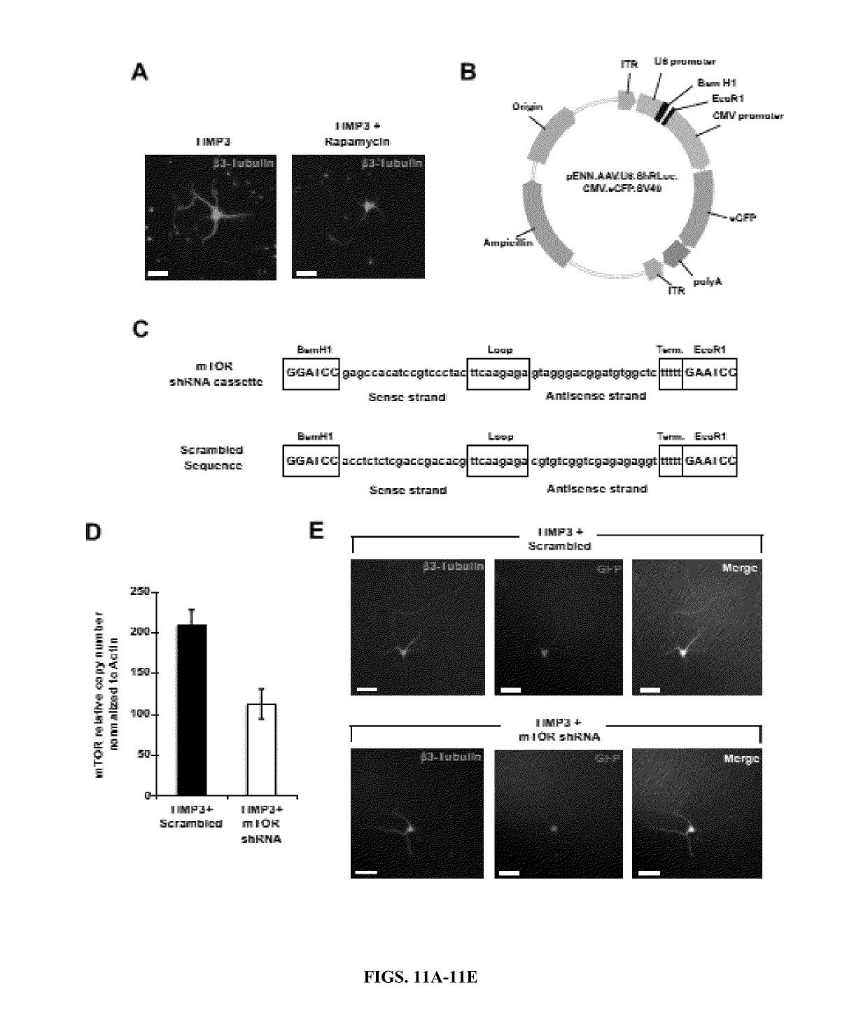

FIGS. 11A-11E are graphical representations depicting various data related to embodiments of the invention. (A) Rapamycin attenuation of neurite outgrowth during TIMP3 treatment. Images representative of result. Scale bar: 50 μm. (B) Synthesized strands targeting mTOR and a scrambled (SCR) version of the sequence was cloned into the expression plasmid pENN.AAV.U6.ShRLuc.CMV.eGFP.SV40. (C) ShRNA against mTOR and shSCR cassettes layout and sequences (SEQ ID NO: 1—mTOR; SEQ ID NO: 2—scrambled sequence). (D) Real-time PCR result. shRNA expression results in knockdown of the mTOR message compared to the scrambled control sequence during TIMP3 treatment. Hippocampal neurons were cultured for 7 days post-transfection prior to analysis. RNA was collected and the relative levels of mTOR message was compared to actin for both shRNA and the scrambled control sequence. (E) Representative images of hippocampal neurons transfected with the shRNA and Scrambled sequence plasmids show the extent of neurite outgrowth (p3-Tubulin). Antibodies against GFP were used to identify transfected neurons. Scale bar: 50 μm.

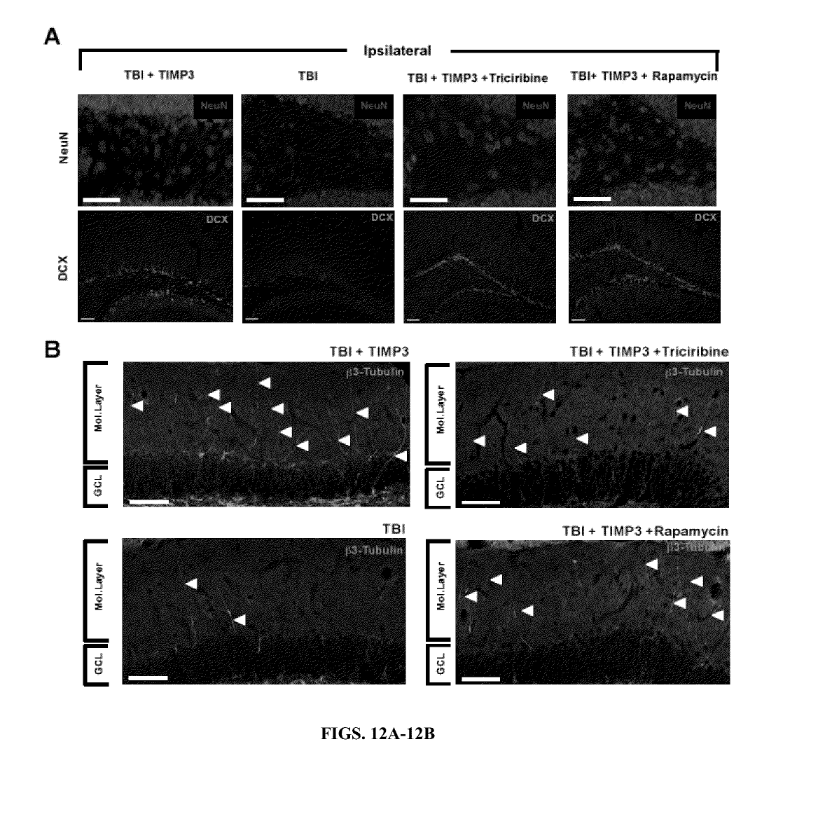

FIGS. 12A-12B are graphical representations depicting various data related to differential effects of Akt-mTOR pathway inhibitors on the protective effects of IV TIMP3 7 days post-TBI. (A) Upper Panel. NeuN staining (Red) of post-mitotic mature neurons showing protective effect of IV TIMP3 versus TBI alone is subverted by triciribine but not rapamycin. Scale bar=100 μm. Lower Panel. DCX staining (Green) of neural progenitors/immature neurons showing the protective effect of IV TIMP3 versus TBI alone is not significantly decreased by either triciribine or rapamycin treatment. Scale bar=100 μm. (B) Staining of 33-Tubulin (Green) showing the protective effects of IV TIMP3 versus TBI alone on dendritic branches in the molecular layer of the dentate gyrus is subverted by triciribine and rapamycin. Arrows indicate branches>50 μm. Scale bar 100 μm.

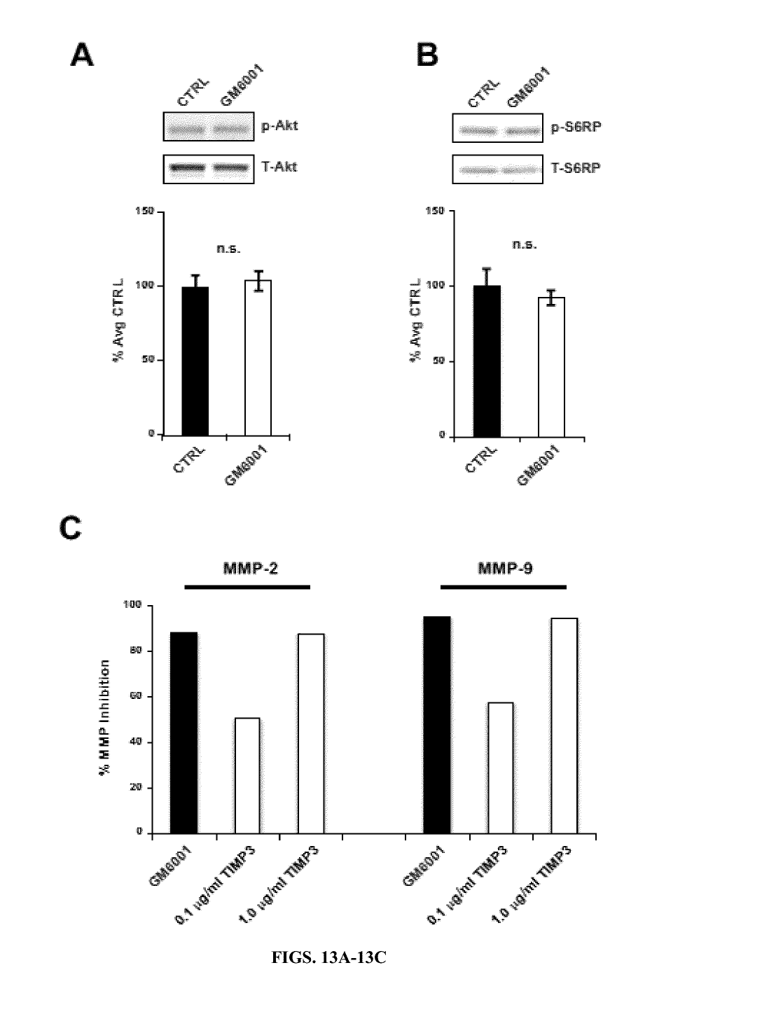

FIGS. 13A-13C are graphical representations depicting various data related to TIMP3 induced activation of the Akt-mTOR pathway. Activation is not dependent on MMP inhibition. Differentiated SH-SY5Y cells were treated with 10 μM of broad-spectrum MMP inhibitor GM6001 for 15 minutes. (A) Western blot. Treatment GM6001 did not result in increased phosphorylation of Akt (Ser⁴⁷³) or (B) S6RP (S^{235/236}). n=4, mean±s.e.m. (C) Sensolyte™ FRET assay. Dose response of TIMP3 demonstrates that 1 μg/ml TIMP3 can fully inhibit MMP-2 and MMP-9 at the concentration observed to activate Akt and mTOR pathways.

Results

Intravenous TIMP3 Abrogates Hippocampal-Dependent Neurocognitive Decline Following TBI.

As initial studies demonstrated protective effects of intravenous (IV) delivered TIMP3 on BBB permeability post-TBI, it was desirable to determine if IV TIMP3 could also attenuate neurocognitive decline. Using a mouse model of controlled cortical impact (CCI), whether post-injury TIMP3 administration could attenuate a performance decline in tasks that strongly involve the hippocampus was examined. TIMP3 was delivered in 3 temporally separated doses followed by testing on the Elevated Plus Maze (EPM) (Zhang et al., Behav Brain Res. 2013, 1-10), Morris water Maze (Gerlai, Behav Brain Res. 2001, 125(1-2):269-277) and context-dependent fear discrimination) (Gerlai, Behav Brain Res. 2001, 125(1-2):269-277) tasks. Experiment design is illustrated schematically in FIG. 1A.

One of the major debilitating neuropsychological aspects of TBI is the formation of increased anxiety. Classically the hippocampus is known to be associated with learning and memory but it is also one of the principal structures, along with the amygdala and the pre-frontal cortex that is involved in regulating stress and anxiety. Understanding the role of the hippocampus is further complicated by the knowledge that the dorsal and ventral divisions of the formation are differentially polarized with respect to anxiety. Rodent studies using the EPM demonstrate that lesions of the dorsal hippocampus are anxiogenic whereas lesions of the ventral hippocampus are anxiolytic. As human hippocampal deficits correlate with increased anxiety disorders, TBI alone (no treatment) was compared to TBI+TIMP3 mice in an elevated plus maze (EPM) (Schwarzbold et al., J Neurotrauma, 2010; 27(10):1883-1893). Observations that both TBI alone mice and TBI+TIMP3 mice entered the closed arms of the EPM with comparable frequency indicate that there are no differences in general movement capacity of the mice between the two groups (FIG. 1B, Left panel). However, TBI mice entered the open arms significantly less frequently than TIMP3 treated mice, thus indicating that TIMP3 treated mice display decreased post-TBI anxiety-like behavior (FIG. 1B, Right panel).

Next spatial learning and memory was examined using an abbreviated version of the Morris water maze task (Guzowski, Proc Natl Acad Sci USA, 1997; 94(6):2693-2698). The two groups did not significantly differ in their ability to learn the platform location during training (FIG. 1C, Left) nor did they differ during the short-term probe to recall the location of the hidden platform conducted 30 minutes after the last training session (data not shown). The long-term memory probe, conducted 24 hours after the last training session demonstrated that the two groups did not significantly differ in the latency to first platform crossing (FIG. 1C, Middle), however TIMP3 treated mice did demonstrate a significant increase in the total number of correct quadrant platform crossings (FIG. 1C, Right). We further examined the ability of the mice to localize the location of the platform during the long-term memory probe by analyzing the number of crossings of a series of concentric rings surrounding the platform (1×, 2×, 3×, and 4× the platform size). Upon examination it was found that TIMP3 treated mice spent more time around and crossing the concentric circles than TBI alone mice (FIG. 1D). This latter result suggests that TIMP3 treatment was able to prevent some long-term memory deficits following TBI.

Context-dependent fear discrimination is a more complex form of learning that requires the ability to form distinct memories about two environments (a shock-associated and a safe context). Performance in this task has been shown to correlate with the number of DCX positive cells and the rate of neurogenesis in the hippocampus. During training (Day 1) neither TBI alone nor TBI+TIMP3 mice discriminated between the two contexts, however on the test day (Day 2) TIMP3 treated mice were able to discriminate between the two environments unlike TBI alone mice (FIG. 1E). Considering the association between DCX positive neural progenitors and context discrimination, these findings suggest that enhanced context discrimination in TIMP3 treated mice may have resulted from survival of DCX neurons. Taken together, all three neurocognitive tests demonstrate that TIMP3 is able to attenuate TBI-generated hippocampal-dependent deficits.

Intravenous TIMP3 Prevents TBI-Induced Loss of Post-Mitotic Neurons and Neural Progenitor Cells in the Dentate Gyrus of the Hippocampus.

Since behavioral testing demonstrated that IV TIMP3 attenuates immediate and long-term TBI-induced cognitive deficits, the inventors sought to determine whether TIMP3 has direct neuroprotective effects, aside from its effects on BBB permeability post-TBI. Using an infra-red tagged version of TIMP3, it was demonstrate that IV TIMP3 administered post-TBI can penetrate into the ipsilateral hippocampus (FIG. 2A), allowing for target coverage and contact of exogenously delivered TIMP3 with cells of the hippocampus. Subsequently, the inventors sought to determine the effects IV TIMP3 post-TBI on hippocampal neuronal populations. TBI has been shown to cause death of both post-mitotic mature neurons and proliferating neural progenitors. The hilus of the dentate gyrus is home to a heterogeneous population of GABAergic inhibitory interneurons that filter information from the granule cell layer before transmission onto the CA3 region. Loss of these interneurons disrupts hippocampal circuitry and causes neurocognitive deficits. Staining of coronal tissue sections through the damaged dorsal hippocampal for the neuronal specific cell body marker NeuN demonstrated a significant reduction in hilar neurons that was abrogated by IV TIMP3 treatment (FIG. 2B). There were no significant differences on the contralateral side for either treatment (FIG. 9A). Although Witgen and colleagues reported a 40% reduction in granule neurons one week post-TBI using designed-based stereology, no appreciable differences were observed at the same time-point in the mouse CCI model with staining of serial tissue sections. To further examine the effects of TIMP3 on neuronal cells the presence of neural progenitors was quantified by DCX staining. Neural progenitors exist in the subgranule zone (SGZ) of the hilus and give rise to DCX positive-immature neurons that then migrate into the granule cell layer and integrate into the brains neural circuitry. Staining of ipsilateral sections for DCX revealed a significant loss of DCX positive cells post-TBI that was attenuated by IV TIMP3 treatment (FIG. 2C). There were no significant differences on the contralateral side for either treatment (FIG. 9B).

TIMP3 Activates Akt-mTORC1 Signaling in Neurons In Vitro.

Having determined that TIMP3 has a protective effect on hippocampal neurons, the inventors sought to further understand this observation at the molecular level. An initial goal was to delineate direct neuronal effects from possible indirect effects of TIMP3 on endothelial integrity and BBB permeability. To determine this directly treated neurons in vitro with TIMP3 and simultaneously assessed 39 unique signaling nodes utilizing Pathscan™ antibody signaling arrays from Cell Signaling Technologies (Danvers, Mass.). Treatment of differentiated SH-SY5Y cells, a neuronal-like human cell line, with TIMP3 for 15 minutes resulted in a significant activation of the pro-survival kinase Akt (FIG. 3A). To confirm the observation and exclude the possibility of a false-positive result, the experiment was repeated with analyses performed by western blot (FIG. 3B) and by Phosflow™ cytometry (FIG. 3C), which allows for evaluation of intracellular phosphorylated proteins. Both techniques confirmed initial findings. To relate these findings to our mouse model the inventors repeated the observations using primary hippocampal neurons as disparity in results have been found for cell lines versus primary cultures. Pathscan analysis on homogenates of TIMP3 treated hippocampal neurons also determined that TIMP3 directly activates Akt in these primary cells (FIG. 3D), which was also confirmed by western blot analysis (FIG. 3E).

Activation of Akt is quintessential for the survival of neurons with its many breakpoints against the apoptotic machinery. However, additional hits were noted from the Pathscan arrays downstream of Akt that did not fall into an anti-apoptotic category. One such observation was a terminal branch point of the mTORC1 pathway, S6 Ribosomal Protein (S6RP) (FIG. 3F). Phosphorylation of S6RP has been previously reported as a marker of mTORC1 pathway activation. Increased array signals for phospho-S6RP were observed for both SH-SY5Y cells and isolated primary hippocampal neurons (FIG. 3G), a finding confirmed by western blot in repeat experiments (FIG. 3H). The activation of S6RP has been previously reported to be involved in regeneration of axons following injury and axonal injury is a significant part in the pathology of TBI.

Intravenous TIMP3 Activates the Akt-mTORC1 Pathway In Vivo Post-TBI

Activation of the Akt-mTORC1 pathway post-TBI has been reported. Using the TBI and IV TIMP3 delivery paradigm applied previously, the inventors desired to determine whether TIMP3 could activate the Akt-mTORC1 pathway in vivo in the hippocampus post-TBI. Animals were sacrificed on day 3 of the procedure, one hour after the last TIMP3 injection as in vitro data from hippocampal cultures on the timing of treatment had indicated that this was the point of maximal phosphorylation (data not shown). As shown (FIGS. 4A-4B), Akt and S6RP phosphorylation levels in homogenates from the ipsilateral hippocampi post-TBI were significantly higher following IV TIMP3 treatment than with TBI alone. No differences were observed between treatments on the contralateral side (FIGS. 10A-10B). Previous studies have reported elevated S6RP phosphorylation 24 hours post-TBI. However, at 3 days post-injury significant differences in S6RP phosphorylation between sham and TBI samples were not evident (FIG. 10C). Previous work using a mouse model of 1BI has shown increased S6RP phosphorylation in the hippocampus in microglia and astrocytes suggesting that this was part of the pathology of TBI. Also, in a rat model increased S6RP phosphorylation post-TBI has been observed but in neuronal subfields. Because of these mixed results the location or cell type where the mTORC1 pathway is activated following TIMP3 treatment was desired to be determined. Animals were sacrificed one hour after the last dose of TIMP3 and tissue sections were stained for phosphorylated S6RP. As shown, (FIG. 4C) strong staining for phosphorylated S6RP was observed in the non-neuronal molecular layer of the ipsilateral hippocampus only in TBI mice. This staining, predominantly co-localized to microglia was also present in astrocytes but to a lesser extent (FIG. 10D). Neither sham controls nor TIMP3 treated TBI mice showed this pattern of staining (FIG. 4C). In TIMP3 treated mice, increases in phospho-S6RP were found in neuronal subfields of the dentate gyrus (FIG. 4D) and the CA3 region (FIG. 4E). Interestingly, ipsilateral hippocampi from TBI mice showed an almost complete absence of phospho-S6RP staining in neurons of the dentate gyrus, levels that were below those observed in sham controls (FIG. 4D). Taken together, these results suggest that IV TIMP3 administered post-TBI prevents S6RP activity in microglia and astrocytes and promotes S6RP activity in hippocampal neurons in direct contrast to observations from TBI-alone mice.

TIMP3 Promotes Both Neuronal Survival and Neurite Outgrowth In Vitro.

Activation of the Akt-mTORC1 signaling pathway could potentially result in both neuroprotection and neurite outgrowth. Whether both effects were possible with TIMP3 treatment was determined. As post-TBI hypoxia is known to exacerbate neuronal death, the neuroprotective capability of TIMP3 using a hypoxic chamber was investigated. In the paradigm, exposure of SH-SY5Y cells to 5 hours of hypoxia followed by 18 hours of recovery at normoxia significantly reduced viability as assessed by MTT assay. However, pre-treatment with TIMP3 for 1 hour significantly attenuated cell death (FIG. 5A). As inhibition of Akt causes death of SH-SY5Y cells the role of TIMP3-induced Akt activation through use of specific Akt inhibitors could not be assessed. However, inhibition of the mTOR pathway in neurons has been shown to only result in reduced neurite outgrowth without a decrease in cell survival. The mTORC1 inhibitor rapamycin did not affect the ability of TIMP3 to inhibit neuronal cell death (FIG. 5B). However, at a concentration that completely blocked S6RP phosphorylation, rapamycin pre-treatment resulted in an elevated level of phospho-Akt. This result, due to a feedback loop correlated with a mild but non-significant increase in neuroprotection (FIG. 5B-5C). Taken together these data suggest that TIMP3 has a neuroprotective effect that involves Aid but not the mTORC1 pathway.

Activation of the mTORC1 pathway in injured retinal ganglia neurons has been shown to induce neurite regeneration whereas pathway suppression results in a failure to regenerate neurites. To determine if TIMP3 induced mTORC1 pathway activation could result in neurite outgrowth, a colorimetric neurite outgrowth assay was utilized. Culturing SH-SY5Y cells on semi-porous membranes allows for direct assessment of neurite outgrowth as only neurites can grow through the pores. As shown, treatment of SH-SY5Y cells with TIMP3 resulted in a significant increase in neurite outgrowth. Furthermore, this increase was completely abrogated by co-treatment with rapamycin (FIG. 5D). To visualize this increase in neurite growth on hippocampal neurons neurite chamber slides were used that allow for the plating of cell bodies in one chamber and neurites to grow through channels into the next chamber under a flow gradient. Seven days of culture with TIMP3 resulted in a clear increase in the density and complexity of neurites (FIG. 5E). In order to quantify differences between treatments on individual neurons, sparsely populated neuronal cultures were analyzed following 72 hours treatment with TIMP3. Sholl analysis (see methods above) conducted on images of isolated neurons demonstrated that treatment resulted in a significantly more complex neurite network (FIG. 5F). As with SH-SY5Y, the increase in neurite outgrowth during TIMP3 treatment was significantly attenuated by rapamycin treatment (FIG. 5G), representative images shown in FIG. 11A. To further determine the involvement of mTOR in the enhancement of neurite outgrowth by TIMP3 the effects of shRNA against mTOR was investigated. The sequence chosen had been previously reported to successfully knockdown mTOR in hippocampal neurons. As a control a scrambled version of the sequence (SCR) was also created (FIGS. 11B-11C). Validation experiments using real time PCR demonstrated that the shRNA sequence was capable of reducing mTOR message levels by approximately 50% compared to SCR (FIG. 11D). Following transfection, the neurons were cultured for 7 days before Sholl analysis was conducted. Successfully transfected neurons were identified by GFP expression. As shown in FIG. 5H shRNA against mTOR resulted in a significant decrease in neurite outgrowth compared to SCR (representative images shown in FIG. 11E). Taken together these results suggest that the mTOR pathway is critical for the effects of TIMP3 and that both rapamycin and shRNA are equivocal in the extent of their impact on TIMP3 promoted neurite outgrowth.

Intravenous TIMP3 Preserves Neuronal Projections In Vivo in the Molecular Layer of the Dentate Gyrus Following TBI.

Given the observations on neurocognitive preservation (FIG. 1), neuronal signaling in vitro and in vivo and (FIGS. 3 and 4), neurite outgrowth in vitro (FIG. 5) and the studies reporting mTORC1/S6RP activation leads to neurite outgrowth the inventors sought to determine how IV TIMP3 treatment affects neurite outgrowth and neuronal connections in vivo post-TBI. To assess the effects of IV TIMP3, western blot analysis was conducted to determine protein levels of the neuronal specific cytoskeletal marker β3-tubulin, a marker previously used to assess in vitro neurite outgrowth (FIG. 5D-H). Homogenates from the ipsilateral hippocampi 7 days post-TBI showed a small but consistent and significant reduction in the total hippocampal level of β3-tubulin. This reduction was attenuated by IV TIMP3 treatment (FIG. 6A). To determine whether this was generalized to all regions of the hippocampus or was specific to any sub-region we conducted tissue staining for β3-tubulin. Although subtle changes in other subfields are not ruled out, only specific changes in the molecular layer above the dentate gyrus was observed (FIG. 6B). Quantification of the number of observable dendritic branches in the molecular layer proximal to injury revealed a significant reduction post-TBI that was attenuated by IV TIMP3 treatment. Thus, TIMP3 treatment preserves cytoskeletal morphology and circuitry of the hippocampus that could potentially improve hippocampal function and neurocognition following brain injury.

Pharmacological Inhibition of mTOR and Akt Differentially Subvert the Protective Effects of IV TIMP3.

Since observations in vitro demonstrated that TIMP3 mediated activation of the Akt-mTORC1 pathway can promote neuronal survival and neurite outgrowth, the latter of which could be subverted by rapamycin (FIG. 5), the inventors sought to determine if these findings translate in vivo in TBI. Although Akt could not be inhibited with triciribine in vitro, in vivo studies have shown that inhibition of both mTORC1 (with rapamycin) and Akt (with triciribine) can be achieved with systemic administration.

The biological model described herein (summarized in FIG. 7A) suggests that Akt inhibition would inhibit survival of mature neurons (NeuN staining), neuronal progenitors (DCX staining) and the preservation/reinnervation of neuronal projections ((33-tubulin staining) by TIMP3 whereas mTORC1 inhibition would only inhibit the preservation/reinnervation of neuronal projections. To address these hypotheses, TBI+TIMP3 experiments were repeated with 3 IV injections of 60 μg/kg TIMP3 (1 hr, 24 and 72 hours post-TBI) but modified the design to include intra-peritoneal injections of either 1 mg/kg triciribine or 1 mg/kg rapamycin 30 minutes prior to each IV TIMP3 delivery. As before, animals were sacrificed 7 days post-TBI and tissue sections were stained for markers NeuN, DCX and β3-tubulin.

The results from the ipsilateral hippocampus are summarized in FIG. 7B. The results are presented as percent change from TBI+TIMP3 to emphasize the effect of the inhibitors. In inhibitor treated mice significant alterations in NeuN, DCX and β3-tubulin staining were found. As predicted, survival of NeuN positive mature neurons was attenuated by triciribine (white bars) but was unaffected by rapamycin treatment (checked bars). However, the effects of the inhibitors on survival and presence of DCX positive neuronal progenitors was not consistent with our hypothesis. The lack of change in DCX positive staining with triciribine is possibly due to a differential sensitivity of neural progenitors to Akt inhibition compared to the more vulnerable post-mitotic neurons. In addition, the significant increase in staining for DCX positive neuronal progenitors with rapamycin (an indication of enhanced proliferation) has been previously reported and is therefore consistent with successful delivery of rapamycin.

Also consistent with this hypothesis was the observation that both triciribine and rapamycin subverted the preservation/reinnervation of neurites in the molecular layer of the dentate gyrus (β3-tubulin staining) (representative images for the effects of the inhibitors on NeuN, DCX and β3-tubulin staining are shown FIG. 12). No significant changes in any of the markers were observed on the contralateral side (data not shown). Taken together, these observations suggest that the Akt-mTORC1 pathway is critically involved in the effects of TIMP3 on neurons and their projections in vivo post-TBI.

Discussion

In this study, using an established CCI mouse model of TBI, it is demonstrated that IV TIMP3 attenuates neurocognitive deficits and hippocampal neuronal cell loss post-TBI. Likely underlying the preservation of neurocognitive ability is our finding that TIMP3 treatment preserves several key features of the hippocampal cyto-architecture that are otherwise lost in TBI. IV TIMP3 treatment is associated with the preservation of vulnerable neuronal populations and neurites in the molecular layer of the dentate gyrus. Through a multi-faceted approach it is demonstrated that TIMP3 treatment activates signaling cascades in neurons, specifically the Akt-mTORC1 pathway, that provide protection against TBI-induced insults such as hypoxia. The in vitro and in vivo data suggest that the Akt-mTORC1 signaling cascade is a critical mediator of the effect of TIMP3 on neuronal survival and preservation of neurite integrity post-TBI. Moreover, it was found that pharmacological and genetic inhibition (with shRNA against mTORC1) of these signaling cascades can subvert the effects of TIMP3. This is the first study to report that intravenous TIMP3, administered post-TBI, results in neurocognitive improvement and neuroprotection. Previous study demonstrating the protective effects of IV TIMP3 on the BBB and the current study elucidating the direct neuroprotective effects of TIMP3, suggest that TIMP3 may have pleiotropic beneficial effects and therapeutic potential in the treatment of patients suffering from traumatic brain injury.

In this study it is demonstrated that IV TIMP3 delivered in three temporally separated doses post-TBI can improve neurocognitive functioning in hippocampal-dependent tasks: the Elevated Plus Maze, the Morris-Water Maze and context-dependent fear discrimination. As damage to the hippocampus is known to generate anxiety in both humans and mouse models of 1BI, initial result that TIMP3 treatment was able to reduce post-TBI anxiety in the Elevated Plus Maze indicated that TIMP3 could be neuroprotective. Furthermore, the Morris Water Maze result suggests that TIMP3 was also able to prevent some long-term post-TBI memory deficits as TIMP3 mice had correctly recalled the location of the platform. Interestingly, the most potent effects of the treatment were found to be improvement in context-dependent fear discrimination. Recent studies have shown that integration or preservation of newborn neurons (neural progenitors) into the hippocampal circuitry is critically involved in the formation of certain kinds of learning and memory, specifically context discrimination. Furthermore, selective elimination of hippocampal progenitor cells impairs the ability of rodents to discriminate in these paradigms. Since TIMP3 attenuates the loss of neural progenitors (DCX positive cells) in the dentate gyms it is plausible that this protective effect contributes to the observed improvement in context-dependent fear discrimination observed in TIMP3 treated TBI mice

To better understand the neuroprotective mechanism underlying the potent beneficial effects of TIMP3 on neurocognition, the effects of TIMP3 on signaling pathways associated with neuronal survival were investigated. In the study it has been shown that TIMP3 treatment results in increased phosphorylation of both Akt and a terminal branch of the Akt-mTORC1 pathway, S6RP. A significant difference between sham and TBI injured mice without treatment was not observed. This differs from some previous studies that have reported activation of these pathways in the hippocampus within the first 24 hours of TBI but differences between the studies and their design have led to a lack of a consensus on whether pathway activation is harmful or beneficial to recovery. Previous reports show significantly increased phospho-S6RP in neurons of the dentate gyrus, CA3 and CA1 sub-regions suggestive of a repair mechanism whereas other reports the increase to be in microglia and astrocytes and not neurons suggestive of a role in inflammation. The inventors also found an increase in microglial staining for phospho-S6RP post-TBI that was absent with IV TIMP3 treatment, suggestive of an anti-inflammatory effect of TIMP3 that could contribute to the neuroprotection observed in treated TBI mice. It was hypothesized that the significance of phospho-S6RP activation in TBI is cell type specific. The in vitro and in vivo studies clearly demonstrate that activation of the Akt-mTORC1 pathway in neurons is associated with neuroprotection and neurocognitive improvement and critical for the noted effects of TIMP3.

At the cellular level, the in vivo studies demonstrate that IV TIMP3 treatment either preserves or increases neurite outgrowth thereby preventing loss of connectivity in the dentate gyrus post-TBI. This finding potentially contributes to the attenuated neurocognitive decline imparted by IV TIMP3 treatment. These data are in agreement with previous studies that have linked the mTORC1 pathway to the generation and/or repair of neuronal projections and connections. Correct information processing depends on a balance between neuronal excitation and inhibition. On a molecular level, TBI is known to render the dentate gyrus hyper-excitable due to a loss of inhibitory interneurons. It is shown here that TIMP3 preserves both neuronal projections and inhibitory hilar neurons in the dentate post-TBI which could help preserve a normal balance in signaling and connectivity, thereby potentially preventing the deleterious consequences of TBI induced hyperexcitability.

Results from both the in vitro and in vivo experiments have allowed us to hypothesize that the observed activation of the Akt-mTOR pathway, that accompanies TIMP3 treatment is the mechanism that imparts neuroprotection and neurite outgrowth. To this end, the inventors sought to determine if inhibition of the pathway could overcome some of these beneficial effects noted for TIMP3 in TBI. Results obtained in vitro demonstrate that pharmacological inhibition of the mTORC1 pathway with rapamycin abrogates the effects of TIMP3 on neurite outgrowth in primary hippocampal neurons. Furthermore, inhibition of mTORC1 with shRNA recapitulates the effects of rapamycin. Interestingly, the same effects were reproduced in TBI mouse model. In these studies, the readout of TIMP3 function was survival of NeuN positive hilar interneurons and hippocampal dentate gyrus neurite outgrowth post-TBI. It was anticipated that inclusion of the Akt inhibitor triciribine would result in a decrease in the survival of neuronal populations and neurites in the molecular layer, since Akt is upstream of mTOR, whereas rapamycin would only affect neurite outgrowth. This was indeed the case. The results with DCX positive neuronal progenitors were not as robust and we did not see an effect on these cells with triciribine. It was observed that triciribine, despite its expected effects on β3-tubulin and NeuN staining did not result in a significant decrease in DCX staining with TIMP3 treatment. Given the limited understanding of the role of Akt activation on DCX staining, the only hypothesis to put forth is that the DCX positive neural progenitors are not as sensitive or vulnerable to Akt inhibition as NeuN positive post-mitotic neurons.

The working biological model of the many beneficial effects of TIMP3 in preventing neurocognitive decline following TBI is summarized in FIG. 8. The specific effects on neurons, although important are by no means the only neurotrophic observation. Suppression of microglia activity and the attenuation of BBB permeability are also key elements necessary to long-term recovery in TBI, all of which may be contributing to the protective effects of IV TIMP3 on neurocognition post-TBI. There is now growing body of evidence to suggest that other neurological diseases have BBB permeability components to their pathology and TIMP3, with its pleiotropic effects may be applicable to many of the neurological disorders that challenge our society.

These studies not only provide mechanistic insight into the biological effects of TIMP3 but also the therapeutic effects of MSCs in TBI. Although there are over 300 trials listed on ClinicalTrials.gov for MSCs, little is know about their exact mechanisms of action. This is critical for full translation and commercialization of MSCs in TBI patients. This disclosure demonstrates that TIMP3 has potent neuroprotective effects that likely involve an unknown upstream receptor triggering the Akt-mTORC1 pathway in neurons. Current knowledge of the mechanism of action of endogenous TIMP3 is that it differentially interacts and inhibits members of the Matrix metalloproteinases (MMP) family. However, these studies demonstrate that the direct protective effects of TIMP3 on neurons are not a consequence of this inhibition as a broad-spectrum MMP inhibitor, (GM6001) did not enhance Akt or S6RP phosphorylation in neurons (FIGS. 13A-13B). Furthermore, we demonstrate that complete MMP inhibition of TIMP3 targets: MMP-2 and MMP-9, was achieved with GM6001 but also TIMP3 at a concentration that activated the Akt and mTOR pathways and protected neurons from hypoxia induced cell death (FIG. 13C). Taken together our collective data strongly suggests that TIMP3 has protective effects on the neurovascular unit as a whole and can mitigate the multiple deleterious effects of TBI including cerebral edema, neuronal loss, neuroinflammation and most importantly neurocognition. To date the multiple clinical trials that have been run in TBI have unfortunately failed to demonstrate efficacy. Investigation into the reasons behind this have revealed trial design and the pleiotropic nature of the disease with multiple therapeutic targets to be potential causes for failure. It is conceivable that multiple therapeutic targets may need to be addressed at the same time to achieve mitigation of disease symptoms and long-term improvement of outcomes in TBI. The data suggest that TIMP3 may be a worthy candidate for further investigation due to its ease of intravenous delivery and its ability to address both neuroprotection and blood brain barrier compromise simultaneously in TBI.

### Example 2

### Neuroprotective Effect of Wnt3a

This example demonstrates that that intravenous delivery of recombinant Wnt3a (IV-rWnt3a) post-TBI mimics the neuroprotective and neurogenic effects of IV-MSCs.

In the present study, the inventors sought to investigate the effects of IV-MSCs on hippocampal neurogenesis and neuronal survival after TBI and determine if a soluble factor potentially mediates these effects. It was found that post-TBI BI IV-MSC treatment promotes the survival of newborn neuronal progenitors, enhances neurogenesis and increases the dendritic arborization of these new neurons. Exploration into possible soluble factor involvement revealed that IV-MSCs produce Wnt3a and increase circulating and hippocampal Wnt3a levels with concurrent activation of the Wnt/β-catenin signaling pathway in the hippocampus. In previous studies with MSCs and soluble factors, it was shown that MSCs modulate Wnt protein production when in contact with endothelial cells. In this study it is demonstrated that intravenous delivery of recombinant Wnt3a (W-rWnt3a) post-TBI mimics the neuroprotective and neurogenic effects of IV-MSCs. Finally, behavioral studies show that IV-rWnt3a improves the performance of TBI mice in cognitive tasks known to be sensitive and dependent on neurogenesis.

The following experimental methods and procedures were utilized.

Methods

Culture of Human Mesenchymal Stem Cells

First passage human MSCs and growth media were purchased from Lonza (Walkersville, Md.). The cells were maintained in MSC growth medium (MSCGM) in 75 cm² flask and in a humidified incubator at 37° C. with 5% CO₂. Only cells with passage numbers 3-7 were used for experiments.

Animal Model of TBI

8 week old male C57BL/6 mice were purchased from Harlan Laboratories (Indianapolis, Ind.). The animals were housed in a 12-h light/dark cycle with ad libitum access to food and water. All experimental procedures were approved by the Institutional Animal Care and Use Committee of UTHealth at Houston and were conducted in accordance with the recommendations provided in the NIH Guide for the Care and Use of Laboratory Animals. A standard protocol and a controlled cortical impact (CCI) device (Pittsburgh Precision Instruments, Pittsburgh, Pa.) were used to generate brain injuries as previously described.

In vivo Cell and Recombinant Mouse Wnt3a Administration

At 2 and 24 h after injury, either 1×10⁶ MSCs, 400 ng of recombinant mouse Wnt3a (R&D Systems, Minneapolis, Minn.) or vehicle control (phosphate-buffered saline, PBS) per 25 g mouse was administered via tail vein injections in a total volume of 100 μL. Cells were harvested via trypsinization and washed twice in PBS before in vivo administration.

Immunohistochemistry

Immunohistochemistry and confocal microscopy quantification was performed as previously described with minor modifications.

Dendritic Morphology Analysis

Analysis for the dendritic growth and complexity of dentate gyrus neurons were performed as previously described. Briefly, confocal images of DCX+ cells were taken and number of cells were counted as above described and the value is presented as letter a. The next several values (b, c, d) represent the number of dendrites at distinct spans from the cell bodies layer, which are the number of primary, secondary and tertiary dendrites, respectively. They are quantified by manually tracing 3 drawn lines and marking dendrites along their paths. Line 1 is drawn right above the cell bodies and Line 2 and 3 are drawn along the outer edge of GCL and in the middle of molecular layer, respectively. Calculations of dendritic length, branching points are calculated as the following:

Dendritic Length=(c×distance between line 1 and 2)+(d×distance between line 2 and 3)

Branching points=(c+d)/a.

Parameters for levels of complexity are:

Np: The level of primary dendrite sprouting (b/a).

Ns: The level of secondary branching (c/b).

Nt: The level of tertiary branching from secondary dendrites (d/c).

RNA extraction and quantitative RT-PCR

Total RNA extraction and Taqman qPCR were performed as previously described.

Western Blotting

Hippocampus taken from the injured side was harvested and homogenized in 2× cell lysis buffer (Cell Signaling) containing 1 mM of PMSF and other protease and phosphotase inhibitors. 40 μg of tissue lysate was resolved on 4-12% of Criterion XT™ Bis-Tris Gel (Bio-Rad, Hercules, Calif.) and transferred onto PVDF membrane. For mouse serum samples, serums were first depleted of albumin and IgG protein with an Aurum Serum Protein™ kit (Bio-Rad) before being loaded onto gel. Blots were blocked in Odyssey Blocking Buffer™ (Li-Cor, Lincoln, Nebr.) and probed with primary antibodies for Wnt3a (R&D Systems) and total and activated β-catenin (Millipore) overnight at 4° C. Blots were then incubated with infrared fluorophore-conjugated secondary antibodies for 3 h at RT, washed and scanned with Odyssey Imaging System™ (Li-Cor). Densitometric readings of immunoreactive bands were obtained using Odyssey Imaging Software™.

Behavioral Assays

Several behavioral tests were applied to evaluate neurocognitive function in TBI mice after IV-MSC treatment as detailed below.

Context Discrimination

Contextual discrimination is a task dependent on intact hippocampal function and was assessed in a manner similar to prior studies conducted by our lab and others. Animals were pre-exposed (without shock) to two contexts with specific shared features (horizontal grid floor, background noise, animal handling to and from the room) and specific differing features (differently spaced grids, scent, cues and floor shape). Animals were given two trials, one in each chamber, each day. Animals were placed in the shock chamber and 178 sec later, a 2 sec, 0.75 mA shock was given. In the safe chamber, animals were free to roam for 3 min and no shock was given. Animals were exposed to the shock and safe chambers once a day for 3 days. Discrimination of the two contexts was assessed by comparing the time spent freezing in each chamber during training.

Novel Object Recognition

The Novel Object Recognition (NOR) task was performed as previously described with minor modifications. The method relies on the observation that rodents spend more time exploring a novel object than an object it has encountered prior. Object recognition was carried out by first exposing the animal to the training chamber in order to habituate it to the environment. Habituation took place over three days by placing the animal in the center of a 100×100 cm box and allowing free exploration for a period of 10 min. On the fourth day, two identical objects were placed in the cage and the animal was allowed to explore for a period of 10 min. Movement in the cage was recorded by a video camera, and the time spent exploring each object scored by two independent investigators who were blind to the treatment groups. Twenty-four hours after the training, the animal was reintroduced into the training cage. However, during testing, one of the objects will be replaced by a novel object that had not been previously explored. The time spent exploring the novel object relative to the familiar object was used as an index of memory. The cage and objects were cleaned after each use in order to remove any olfactory cues. The objects used in these experiments were based on preliminary experiments to choose objects that did not have a predetermined bias based on their size, texture, color or geometry.

Statistical Analysis

Unless specified, data were analyzed using a Student's t-test for 2 group comparisons and one-way analysis of variance (ANOVA) for multiple group comparisons. Data in graphs are shown as mean±SD.

### DESCRIPTION OF FIGURES

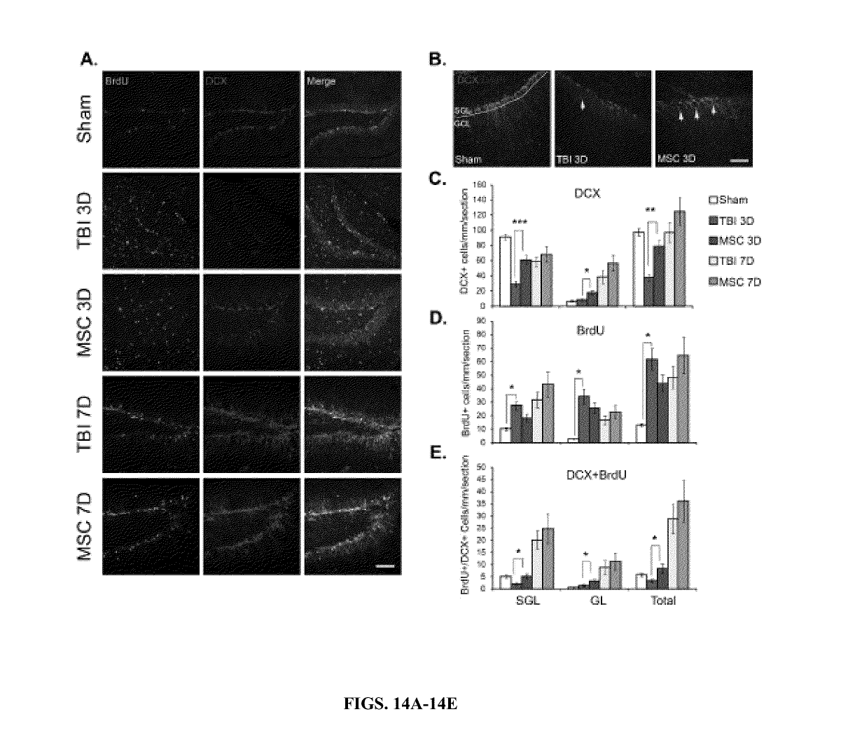

FIGS. 14A-14E are graphical representations depicting various data related to IV-MSC protection of new neurons from TBI-induced loss and enhanced neurogenesis in the ipsilateral dentate gyrus during acute phase post-TBI. Confocal images on coronal sections of mouse dorsal dentate gyrus were stained for BrdU (green), DCX (red) and counterstained with Hoechst for nuclei (blue). (A) Ipsilateral dentate gyrus images reveal loss of new neurons at day 3 day post TBI and rescue by MSC-treatment. Of note is recovery of neurons appears in both TBI alone and MSC-treated dentate gyrus by day 7. Scale bar=100 μm. (B) Close-up view of day 3 post-TBI dentate shows preservation of neurons by IV-MSCs in subgranular cell layer (SGL) and an increase in the granular cell layer (GCL) as indicated by yellow arrows. Scale bar=50 μm (C-E) Quantitation of ipsilateral dentate gyrus (C) DCX+, (D) BrdU+ and (E) DCX+BrdU+ cells. *** p<0.001, ** p<0.01, *p<0.05, data are means±SEM, n=4 mice, 4 sections per mouse. Scale bar=100 μm.

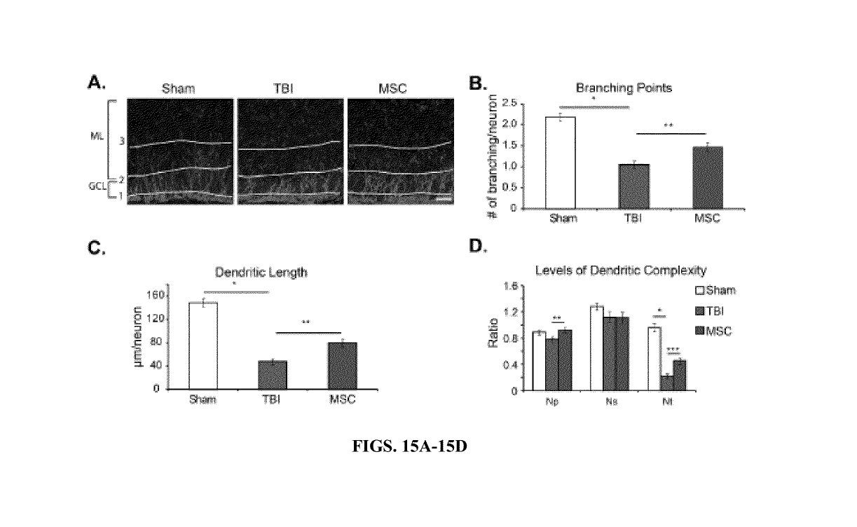

FIGS. 15A-15D are graphical representations depicting various data related to dendritic growth and complexity of ipsilateral hippocampal newborn neurons enhancement by IV-MSCs treatment in TBI. (A) Magnification of DCX staining in the dentate gyrus at day 7 post-TBI highlights dendritic growth of progenitor neurons. Arbitrary lines 1-3 were drawn for quantitation of dendrites as described in methods. Scale bar=50 (B) Quantitation of branching points. * p<0.01, TBI vs. sham; ** p<0.01, MSC vs. TBI. (C) Quantitation of dendritic length. * p<0.01, TBI vs. sham; ** p<0.01, MSC vs. TBI. (D) Levels of dendritic complexity. Np: level of primary dendritic sprouting; Ns: level of secondary branching; Nt: level of tertiary branching from secondary dendrites. * p<0.01, TBI vs. sham; ** p<0.05, MSC vs. FBI; *** p<0.01, MSC vs. TBI. Data are means±SEM, n=4 mice, 4 sections per mouse.

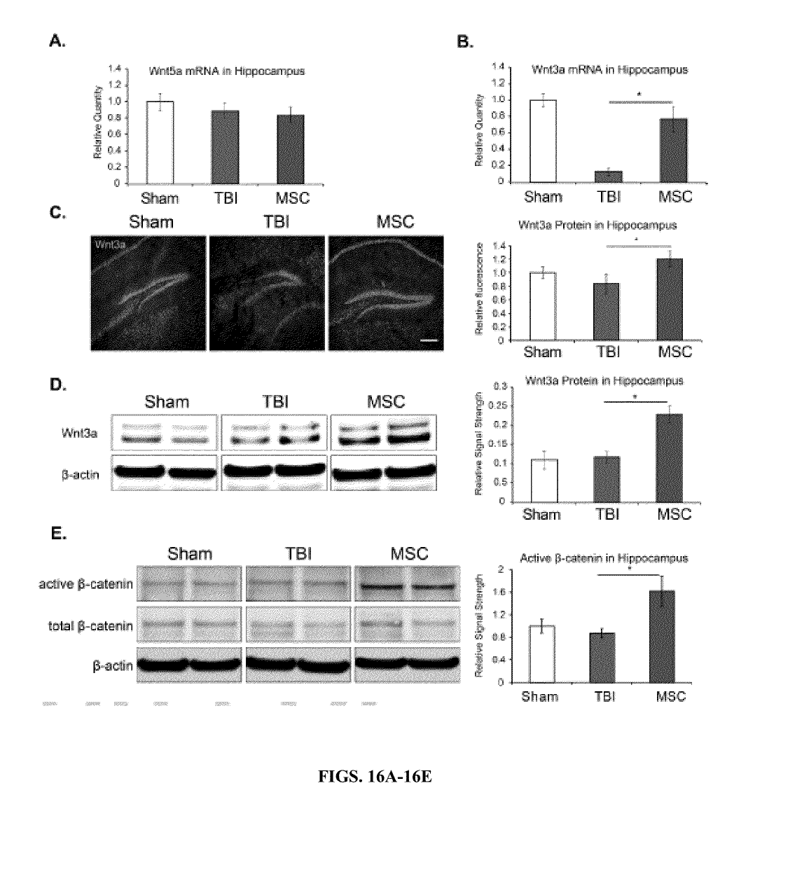

FIGS. 16A-16E are graphical representations depicting various data related to IV-MSC activation of hippocampal Wnt/β-catenin signaling in TBI mice. (A) IV-MSCs did not alter Wnt5a mRNA expression, but (B) increased Wnt3a mRNA expression in the hippocampus of TBI mice. (C-D) IV-MSCs Increased Wnt3a protein expression. (C) Immunofluorescent images show Wnt3a is primarily expressed in the dentate gyrus and CA regions of hippocampus. Fluorescence quantitation indicates increased expression of Wnt3a by IV-MSCs relative to TBI alone. Scale bar=200 μm (D) Western blot for Wnt3a confirms its enhancement of protein expression in hippocampus. (E) Western blot shows IV-MSCs increased unphosphorylated stabilized active β-catenin in the hippocampus. * p<0.05, MSC vs.TBI, data are means±SEM, n=4-5.

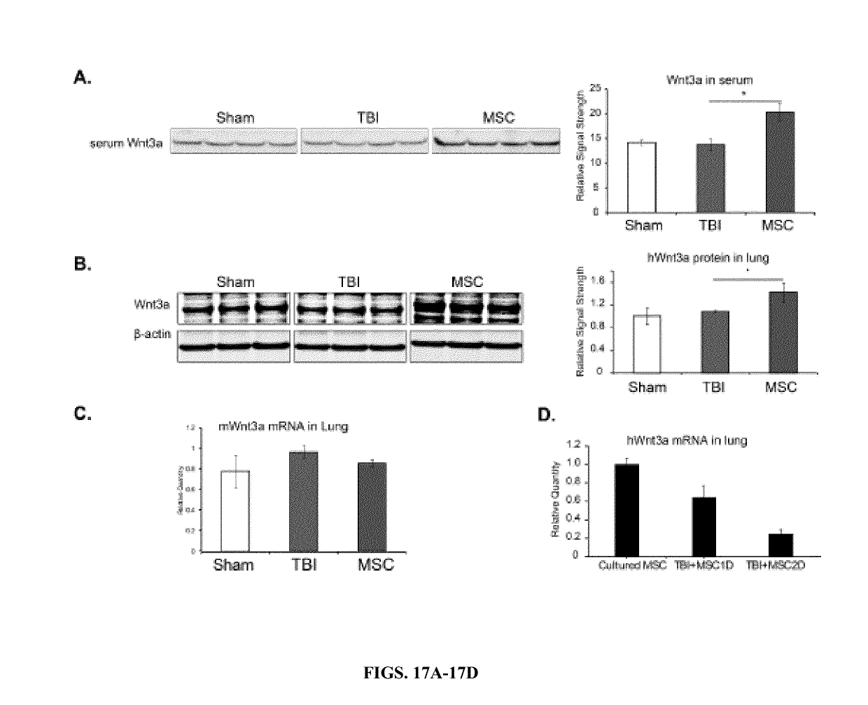

FIGS. 17A-17D are graphical representations depicting various data related to IV-MSC increase of Wnt3a levels in serum and lungs. (A) Increase of systemic Wnt3a in the serum of MSC-treated TBI mice as shown by Western blot. (B) IV-MSCs Increase Wnt3a protein levels in lungs of TBI mice as shown by Western blot. (C) IV-MSCs did not increase Wnt3a mRNA expression in lung at 3-day after TBI. (D) Continued expression of human Wnt3a transcripts in mouse lungs up to 48 h after injection as indicated by qPCR analysis. Quantity of mRNA expression in mouse lung is presented as relative to MSCs in tissue culture.* p<0.05, MSC vs.TBI, data are means±SEM, n=4.

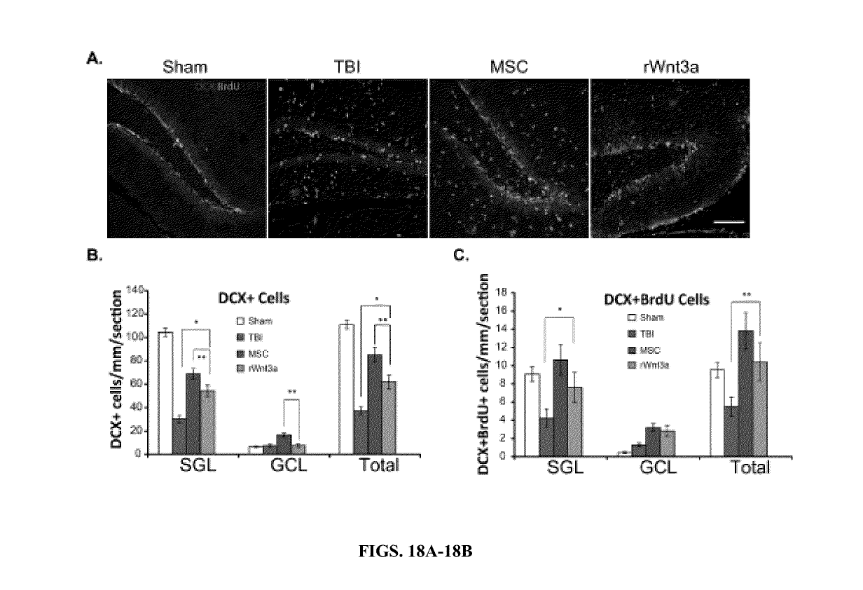

FIGS. 18A-18B are graphical representations depicting various data related to IV-rWnt3a mimicking of the neuroprotective and neurogenic effects of IV-MSCs. (A) Confocal imaging showing status of DCX+ neurons (red) and BrdU proliferation markers (green) in dentate gyrus 3 days after TBI under different treatments. Scale bar=100 μm. (B)-(C) Quantification counts of DCX+ and DCX+BrdU+ cells indicating a partial recapitulation of the neuroprotective and neurogenic effects of IV-in post-TBI hippocampus. * p<0.05, **p<0.01, data are means±SEM, n=4 mice, 4 sections/mouse.

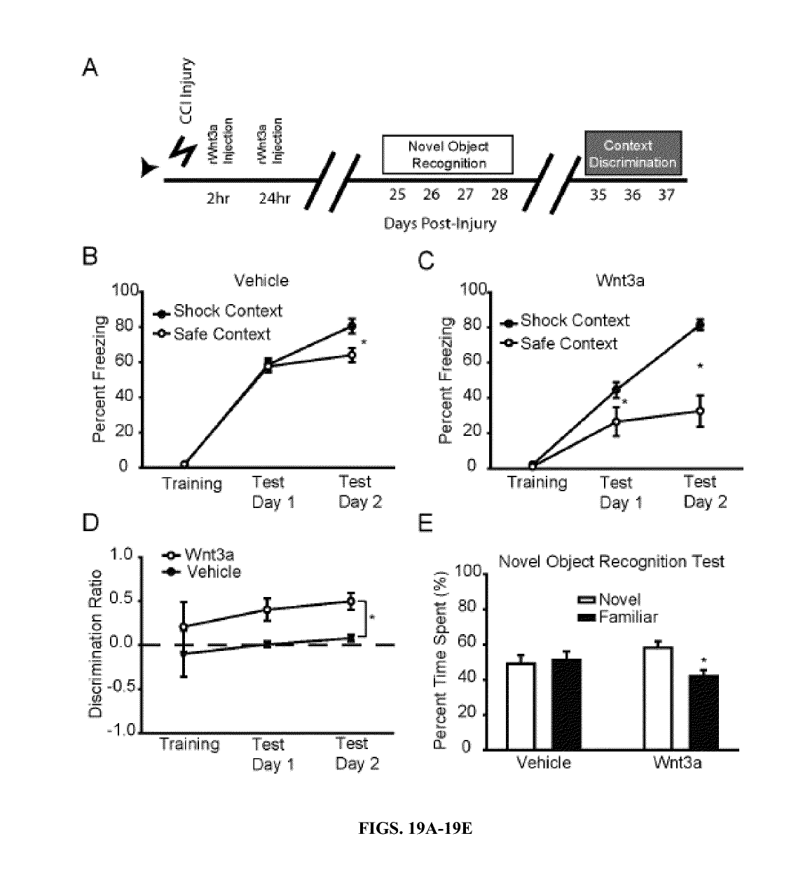

FIGS. 19A-19E are graphical representations depicting various data related to Wnt3a treatment improving cognitive functions in TBI mice. (A) Schematic outline of the experiment. Subjects were given a CCI injury followed by treatment with rWnt-3a at 2 and 24 hours. Twenty-eight days later subjects were tested with novel object recognition followed by a contextual discrimination task. Wnt3a improves the ability to recall memory of familiar objects and discriminate between two contexts. (B) Vehicle treated subjects do not demonstrate an ability to discriminate on the first day of testing, but with further testing they begin to discriminate significantly between the two contexts. (C) Wnt3a improves the brain-injured subject's ability to distinguish between contexts, as indicated by significantly more time spent freezing in the shock context on the first test day. Their time spent freezing in the shock context on the second day of testing continues to increase, while their time spent freezing in the safe context stabilizes. (D) Wnt3a subjects show a greater amount of discrimination between the two contexts as indicated by a discrimination ratio that is closer to 1.0, when compared to vehicle treated subjects. A discrimination ratio of 0 indicates that the subject spent an equal amount of time freezing in both contexts. *p<0.05, data are means±SEM, n=10. (E) Wnt3a improves the mouse performance on novel object recognition test. Wnt3a subjects spent more time on exploration of novel object over familiar one. * p<0.05, data are means±SEM, n=10.

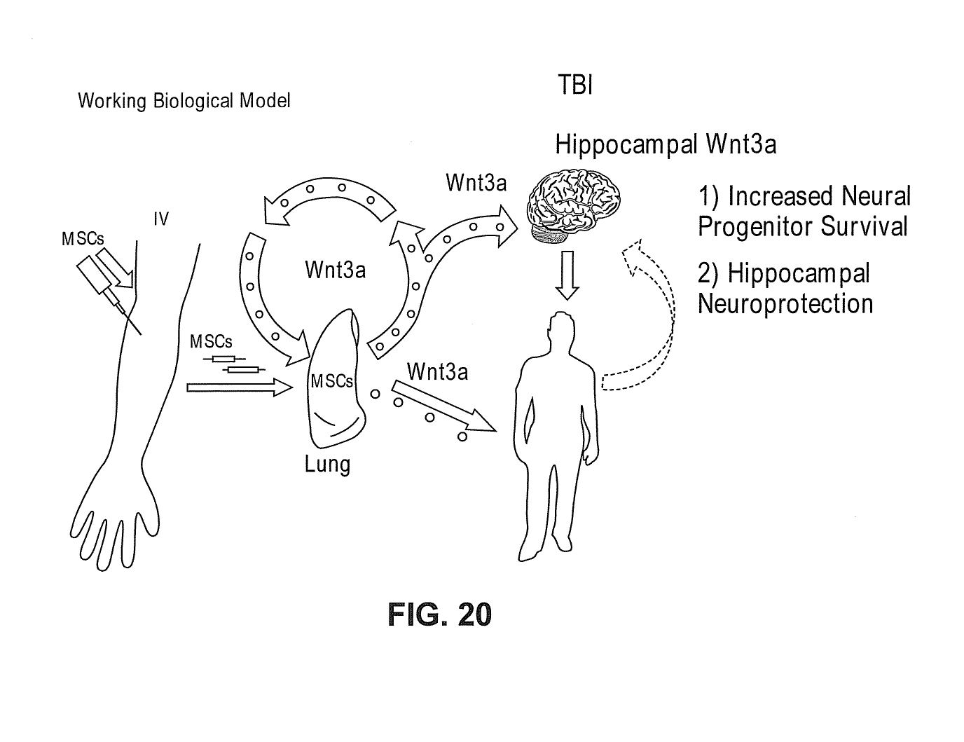

FIG. 20 is a graphical representation depicting the scheme of treatments for the experiments presented in Example 2.

Results

IV-MSCs Promote Hippocampal Neural Progenitor Survival and Neurogenesis in Brain Injured Mice.

Using an established model of controlled cortical impact (CCI) injury, the inventors sought to determine if IV-MSC treatment has regulatory effects upon hippocampal neural progenitor cell survival and growth. Unilateral CCI injury was performed on 8-week old (25-30 gram) C57BL/6 mice and intravenously administered 1×10⁶ MSCs at 2 and 24 h post-TBI. To monitor neurogenesis, BrdU was injected intraperitoneally to the mice 24 h before tissue collection. Whole brain samples were collected at 3 and 7 d post-1BI for immunostaining. FIG. 20 depicts the scheme of treatments for the experiments. Doublecortin (DCX) is a marker for new, immature progenitor neurons. Combined double staining of DCX with the cell proliferation marker BrdU indicates early-stage neurogenesis and proliferation of neural progenitors. In order to accurately analyze and identify the location of neurogenic activity, the innermost cell layers of the dentate gyrus were delineated as the subgranular cell layer (SGL) and the granular cell layer (GCL) as previously described. Consistent with earlier reports, the results presented in FIG. 14A show that compared with sham mice, TBI caused significant loss of DCX+ neurons in the dentate gyrus at day 3 after injury. These changes were found to occur in both SGL and GCL (FIG. 14B). The number of DCX and BrdU double positive (DCX+BrdU+) cells were also reduced (FIGS. 14A & 14E). Post-TBI IV-MSC treatment markedly attenuated the loss of newborn neurons at day-3 post-TBI (FIG. 14). Given the possibility that the loss of DCX+ cells may be due to a decrease in DCX expression and not necessarily be a result of neuronal degeneration or death from injury, Fluoro-Jade C staining was performed which selectively labels degenerating neurons. Similar to that observed using DCX immunohistochemistry, there is a qualitative decrease in the number of degenerating neurons within the ipsilateral dentate gyrus of brain injured mice treated with MSCs compared to those receiving vehicle (data not shown). By day 7 post-TBI, a significant increase of hippocampal neurogenesis was seen as a result of TBI as evidenced by increases in the numbers of both DCX+ and DCX+BrdU+ cells (FIGS. 14A, 14C & 14E). At this time point, there are no apparent differences in cell numbers between the MSC and vehicle-treated control groups at this time point.

In the contralateral hippocampus, DCX+ cells in the GCL slightly increased at day 3 following TBI compared to sham-operated controls, with significant increases in the number of DCX+ and DCX+BrdU+ cells seen in both the GCL and SGL at the 7-day post-TBI time point. In contrast to that seen on the ipsilateral side, treatment with MSCs did not appear to affect the numbers of DCX+ or DCX+BrdU+ cells within the contralateral hippocampus (data not shown).

IV-MSCs Regulate Hippocampal Neural Connectivity by Enhancing Dendritic Growth of Dentate Gyrus Neurons

Dendritic growth of newborn neurons is a fundamental process for synaptic connectivity and functionality. Therefore whether MSC treatment alters dendritic arborization was examined. Although a significant difference between MSCs vs. TBI alone in the number of proliferating neurons at day 7 post-1BI was not observed, a closer examination on the dendritic morphology of DCX+ neurons revealed visible changes between IV-MSC-treated mice compared to TBI alone (FIG. 15A). A recently developed laminar quantification method was used to analyze dendritic growth in 3 categories: average branching points, dendritic length and levels of dendritic complexity. FIGS. 15B-15D show that the number of branch points, overall dendritic length as well as primary sprouting and tertiary branching were significantly reduced as a result of 1BI. Treatment with MSCs reversed such reductions and significantly enhanced dendritic growth and complexity (as assessed by the above) of newborn neurons at Day 7 post-TBI (FIGS. 15B-15D).

IV-MSCs Activate Hippocampal Wnt/β-Catenin Signaling

The Wnt/β-catenin signaling pathway is known to actively participate in regulation of adult hippocampal neurogenesis and MSCs have been reported to augment hippocampal neurogenesis through Wnt signaling in other disease models. As the Wnt family of morphogens consists of 19 known members, previously published microarray results were queried to determine if any of the Wnt family members were induced in MSC-endothelial cell co-cultures. Wnt3a and Wnt5a were identified as possible candidates for the neurogenic effects of IV-MSCs. Therefore the expression levels of Wnt3a and Wnt5a were examined in the hippocampus following IV-MSC treatment of TBI mice. It was determined that the mRNA levels of hippocampal canonical Wnt pathway ligand Wnt3a increased by about 4-fold in TV-MSC treated mice compared to TBI alone. Levels of non-canonical ligand Wnt5a remained largely unchanged between groups (FIGS. 16A & 16B). Wnt3a protein, as determined quantitatively by immunoflourescent staining was also increased (FIG. 16C). These results were corroborated by Western blot analysis which showed increases in Wnt3a protein levels in TBI IV-MSC mice as compared to TBI alone (FIG. 16D). Western blot also revealed that TV-MSCs increased levels of the active, unphosphorylated form β-catenin, which serves as the central downstream transducer in Wnt activation (FIG. 16E). Taken together, these data indicate that TV-MSC treatment activates hippocampal Wnt/β-catenin signaling.

IV-MSCs Increase Wnt3a Levels in Serum and Lungs.

To further elucidate the mechanism by which IV-MSCs might activate hippocampal Wnt/β-catenin signaling, the possible role of MSC-released soluble factors was considered based on prior studies demonstrating that the majority of IV-MSCs are trapped inside the lungs due to a first pass effect. Measurement of serum Wnt3a levels revealed that administration of IV-MSCs increases the levels of circulating Wnt3a in TBI mice (FIG. 17A). There was also a substantial increase in Wnt3a protein expression in the lungs of IV-MSC treated mice (FIG. 17B). Murine Wnt3a levels remained unchanged between groups (FIG. 17C), however, human Wnt3a levels, produced by MSCs in the lungs is maximal at day 1 post injection (FIG. 17D). This shows that MSCs in culture and in the lungs do indeed produce Wnt3a, which maintains substantial levels in vivo up to two days post-injection. These data support the premise that IV-MSCs release soluble factors such as Wnt3a that can contribute to their therapeutic effects in vivo.

Recombinant Wnt3a Mimics the Neuroprotective and Neurogenic Effects of IV-MSCs and Improves Neurocognitive Outcomes Following TBI.

To test the role of Wnt3a in the neuroprotective and neurogenic effects of IV-MSCs, TBI mice receiving intravenous recombinant Wnt3a (rWnt3a) were compared to those receiving IV-MSCs. FIG. 18 shows that rWnt3a mimicked MSC-mediated preservation of DCX+ neural progenitor cells at day 3 post-TBI. It is of interest to note that the effects of rWnt3a are quantitatively diminished compared to those seen with MSCs, suggesting that other factors released by MSCs or alternate effects of MSCs may also contribute to these effects. This concept is further supported by data (not shown) showing that rWnt3a did not reduce microglia (Iba+) cell numbers in the injured hippocampus as described in past work for MSCs. To examine the effects of IV-rWnt3a on neurocognition, context discrimination and novel object recognition tasks known to require hippocampal neurogenesis were employed.

FIG. 19A provides an overview and the chronological order of the experiments. Contextual fear discrimination is a task that requires mice to differentiate between two similar but distinct contexts. Vehicle-treated injured animals require two days of training in order to distinguish between the shock and the safe contexts as indicated by reduced freezing in the safe context (FIG. 19B). By comparison, injured animals treated with rWnt3a were able to discriminate between the two contexts after only a single training trial (FIG. 19C). In addition, rWnt3a treated subjects showed higher level of discrimination than vehicle-treated ones, as indicated by the discrimination ratio (FIG. 19D). The novel object recognition test takes advantage of a mouse's tendency to approach and explore novel objects and requires an intact memory of previously experienced objects. While vehicle treated mice showed no difference in discriminating either familiar or novel objects, rWnt3a-treated mice spent more time in exploring the novel objects than the familiar ones (FIG. 19E). Taken together, these data show that IV rWnt3a mimics the neuroprotective and neurogenic effects of IV-MSCs and improves neurocognitive outcomes following TBI.

Discussion

Newborn neurons in the dentate gyrus are highly vulnerable to TBI and their loss can contribute to hippocampal dysfunction. In the present study, evidence is provided showing that intravenously administered MSCs protect newborn neurons from TBI-induced death and enhance neurogenesis in the dentate gyrus. It was also found that IV-MSCs increase serum levels of Wnt3a, which may promote activation of Wnt/β-catenin signaling in the hippocampus through its entry via the compromised blood-brain bather in TBI animals. These effects of IV-MSCs with rWnt3a, were reproducible supporting a neuroprotective and pro-neurogenic function in TBI animals. Behavioral studies further supported the therapeutic value of Wnt3a in facilitating neurocognitive improvement.

Temporal changes in neurogenesis after TBI have been well studied in recent years. It has been shown that DCX+ progenitor neurons are vulnerable to injury and suffer major loss in the acute phase post-TBI, but later are repopulated by the more injury-resistant nestin+ neural stem cells. The data herein confirm these findings, showing a sharp decline of DCX+ neurons at 3 days post-TBI followed by recovery in their numbers by day 7. Previous studies on intravenously delivered MSCs and related stem cell treatments in TBI have shown improvement in neurological outcomes, likely due to increased levels of neurotrophic and other growth factors that promote recovery and repair. To date there has been no direct evidence of changes in neurogenesis by IV-delivered MSCs during the early stages after TBI in the dentate gyrus, a key structure implicated in functional recovery after TBI. The data herein are novel and demonstrate that IV-MSC treatment can prevent substantial loss of DCX+ neurons while also enhancing their dendritic outgrowth into the GCL and molecular layers of the dentate.

Possible mechanisms underlying the multiple therapeutic effects seen with adult stem cells, including regulation of post-TBI hippocampal neurogenesis, remains under debate. For transplanted MSCs, which are directly administered to the brain, proposed theories have postulated their influence on endogenous neuronal activities and include cell fusion and trans-differentiation with the release of soluble factors. In the case of IV-delivered MSCs, much of our earlier work has ruled out the possibility of direct effects within the brain since MSCs are rarely detectable at the site of injury. The majority of MSCs can be found trapped in the lung due to first pass effects, with a smaller proportion also located in the heart, liver and spleen. Stronger evidence exists implicating soluble factors released from MSCs and other local cells they are directly interacting with, such as pulmonary endothelial cells and splenocytes. For example, intravenously administered MSCs have been shown to reduce inflammation in chemically damaged corneas through secretion of an anti-inflammatory protein.

The canonical Wnt signaling pathway ligand Wnt3a is a principle regulator of adult hippocampal neurogenesis and the Wnt/β-catenin pathway plays a critical role for the differentiation and survival of NSCs/NPCs. Activation of Wnt signaling has been identified in the enhancement of neural regeneration after injury, as the Wnt pathway promotes symmetrical division of NSCs and facilitates tissue repair. Wnt signaling molecules including Wnt(s) ligands are highly expressed in MSCs and in a model of Alzheimer's disease, hippocampal neurogenesis was enhanced by IV-MSCs through activation of Wnt/β-catenin signaling. The inventors found that Wnt3a but not Wnt5a levels were significantly increased in the injured hippocampus and in the serum of MSC-treated mice 3 days post-TBI. Meanwhile levels of activated β-catenin were also increased. Evidence of activation of the Wnt/β-catenin pathway in the hippocampus following MSC treatment suggests that Wnt3a signaling is triggered by MSCs and may be important in mediating the neuroprotective and neurogenic effects of MSCs. Whether circulating Wnt3a directly affects activation of Wnt signaling in the hippocampus is unknown. IV administration of Wnt3a recapitulated the effects of MSCs in the hippocampus, i.e., protection of SGL neurons and enhanced neurogenesis. It is not known whether circulating Wnt3a could cross the intact BBB, but as the BBB is compromised after injury, this may have facilitated its entry.

As currently available antibodies to Wnt3a cannot distinguish between human and murine homologs, it is not possible to determine the species-specificity of the circulating Wnt3a that was observed. In the lungs, expression of murine Wnt3a mRNA in MSC-treated mice was unchanged compared to TBI alone group. However human Wnt3a mRNA was found to be expressed in abundance at 24 and 48 hours post injury in the lungs and is likely produced by IV-MSCs trapped in the lungs. These findings suggest that the increase in circulating Wnt3a we detected may have originated, in part, from the MSCs themselves. Interesting, murine Wnt3a mRNA was increased in the ipsilateral hippocampus, suggesting that within the brain, endogenous expression of Wnt3a is increased by IV-MSCs. One can hypothesize that both MSC-derived Wnt3a and induction of endogenous Wnt3a in the brain contribute to the neuroprotective and neurogenic effects of MSCs after TBI.

Of translational relevance are our findings that IV-rWnt3a, administered acutely after TBI, can improve performance in tasks that have been shown to require hippocampal neurogenesis. New granule neurons generated from hippocampal neurogenesis are thought to play a critical role in a hippocampal computation process referred to as “pattern separation”. Pattern separation has been proposed to enable the hippocampus to distinguish between two similar inputs and separates them into distinct outputs. This process is thought to be essential for certain types of learning and memory such as context discrimination and object recognition. Here it is determined that treatment of TBI mice with IV-rWnt3a improved performance in both contextual discrimination and novel object recognition one month after injury. The one month time point was chosen because prior research has shown that new neurons take 4-6 weeks to become integrated into the hippocampal circuitry. Enhanced neurogenesis and neuroprotection in the hippocampus by IV-rWnt3a may contribute to the improved performance in neurocognitive testing.

The potential therapeutic value and use of IV Wnt3a in TBI offers a new approach for treating neurocognitive dysfunction. The concept of using MSC derived proteins, that can mimic the effects of cell-based therapies, has several advantages. There are significant logistical and practical barriers that arise clinically in the translation of stem cell therapies to patients such as the difficulty of autologous harvest, rejection due to allo-transplantation or the tumorigenic potential of stem cells. Thus the identification and development of a “cell free therapeutic”, a soluble factor(s), that can recapitulate some of the effects of stem cells in TBI could circumvent these barriers in translation and prove invaluable in the treatment of a disease condition with few therapeutic options.

Although the invention has been described with reference to the above example, it will be understood that modifications and variations are encompassed within the spirit and scope of the invention. Accordingly, the invention is limited only by the following claims.

What is claimed is:

1. A method of protecting against neuronal or neural stem cell death comprising administering to a subject having or at risk of having loss of neural function a neuroprotective effective amount of tissue inhibitor of matrix metalloproteinase-3 (TIMP3), thereby protecting against neuronal or neural stem cell death in the subject.

2. The method of claim 1, wherein the subject is a mammal.

3. The method of claim 2, wherein the subject is human.

4. The method of claim 1, wherein administration is by intravenous delivery.

5. The method of claim 1, wherein TIMP3 is administered in a dosage of about 0.0001-1000 mg/kg, about 0.01-1000 mg/kg, or about 0.1-100 mg/kg.

6. The method of claim 1, wherein TIMP3 is administered hourly, daily, weekly, biweekly or monthly.

7. The method of claim 6, wherein TIMP3 is administered at least once or twice daily.

8. The method of claim 1, wherein the subject has or is at risk of having traumatic brain injury (TBI), stroke, Alzheimer's disease, dementia, Parkinson's Disease, amyotrophic lateral sclerosis (ALS), seizure disorder or epilepsy.

9. The method of claim 1, wherein the subject has or is at risk of having a neurodegenerative disorder or condition.

10. A method of enhancing neurogenesis comprising administering to a subject in need thereof a neurogenesis effective amount of tissue inhibitor of matrix metalloproteinase-3 (TIMP3), thereby enhancing neurogenesis in the subject.

11. The method of claim 10, wherein the subject is a mammal.

12. The method of claim 11, wherein the subject is human.

13. The method of claim 10, wherein administration is by intravenous delivery.

14. The method of claim 10, wherein TIMP3 is administered in a dosage of about 0.0001-1000 mg/kg, about 0.01-1000 mg/kg, or about 0.1-100 mg/kg.

15. The method of claim 10, wherein TIMP3 is administered hourly, daily, weekly, biweekly or monthly.

16. The method of claim 15, wherein TIMP3 is administered at least once or twice daily.

17. The method of claim 10, wherein the subject has or is at risk of having traumatic brain injury (TBI), stroke, Alzheimer's disease, dementia, Parkinson's Disease, amyotrophic lateral sclerosis (ALS), seizure disorder or epilepsy.

18. The method of claim 10, wherein the subject has or is at risk of having a neurodegenerative disorder or condition.

19. A method of treating a neurological disorder in a subject in need thereof comprising administering an effective amount of tissue inhibitor of matrix metalloproteinase-3 (TIMP3), thereby treating the neurological disorder in the subject.

20. The method of claim 19, wherein the subject is a mammal.

21. The method of claim 20, wherein the subject is human.

22. The method of claim 19, wherein administration is by intravenous delivery.

23. The method of claim 19, wherein TIMP3 is administered in a dosage of about 0.0001-1000 mg/kg, about 0.01-1000 mg/kg, or about 0.1-100 mg/kg.

24. The method of claim 19, wherein TIMP3 is administered hourly, daily, weekly, biweekly or monthly.

25. The method of claim 24, wherein TIMP3 is administered at least once or twice daily.

26. The method of claim 19, wherein the neurological disorder is traumatic brain injury (TBI), stroke, Alzheimer's disease, dementia, Parkinson's Disease, amyotrophic lateral sclerosis (ALS), seizure disorder or epilepsy.

27. The method of claim 19, wherein the neurological disorder is a neurodegenerative disorder or condition.

28. A method of protecting against neuronal cell death comprising administering to a subject having or at risk of having loss of neural function a neuroprotective effective amount of wingless-type MMTV integration site family, member 3A (Wnt3a), thereby protecting against neuronal cell death in the subject.

29. The method of claim 28, wherein the subject is a mammal.

30. The method of claim 29, wherein the subject is human.

31. The method of claim 28, wherein administration is by intravenous delivery.

32. The method of claim 28, wherein Wnt3a is administered in a dosage of about 0.0001-1000 mg/kg, about 0.01-1000 mg/kg, or about 0.1-100 mg/kg.

33. The method of claim 28, wherein Wnt3a is administered hourly, daily, weekly, biweekly or monthly.

34. The method of claim 33, wherein Wnt3a is administered at least once or twice daily.

35. The method of claim 28, wherein the subject has or is at risk of having traumatic brain injury (TBI), stroke, Alzheimer's disease, dementia, Parkinson's Disease, amyotrophic lateral sclerosis (ALS), seizure disorder or epilepsy.

36. The method of claim 28, wherein the subject has or is at risk of having a neurodegenerative disorder or condition.

37. A method of enhancing neurogenesis comprising administering to a subject in need thereof a neurogenesis effective amount of tissue inhibitor of wingless-type MMTV integration site family, member 3A (Wnt3a), thereby enhancing neurogenesis in the subject.

38. The method of claim 37, wherein the subject is a mammal.

39. The method of claim 38, wherein the subject is human.

40. The method of claim 37, wherein administration is by intravenous delivery.

41. The method of claim 37, wherein Wnt3a is administered in a dosage of about 0.0001-1000 mg/kg, about 0.01-1000 mg/kg, or about 0.1-100 mg/kg.

42. The method of claim 37, wherein Wnt3a is administered hourly, daily, weekly, biweekly or monthly.

43. The method of claim 42, wherein Wnt3a is administered at least once or twice daily.

44. The method of claim 37, wherein the subject has or is at risk of having traumatic brain injury (TBI), stroke, Alzheimer's disease, dementia, Parkinson's Disease, amyotrophic lateral sclerosis (ALS), seizure disorder or epilepsy.

45. The method of claim 37, wherein the subject has or is at risk of having a neurodegenerative disorder or condition.

46. A method of treating a neurological disorder in a subject in need thereof comprising administering an effective amount of wingless-type MMTV integration site family, member 3A (Wnt3a), thereby treating the neurological disorder in the subject.

47. The method of claim 46, wherein the subject is a mammal.

48. The method of claim 47, wherein the subject is human.

49. The method of claim 46, wherein administration is by intravenous delivery.

50. The method of claim 46, wherein Wnt3a is administered in a dosage of about 0.0001-1000 mg/kg, about 0.01-1000 mg/kg, or about 0.1-100 mg/kg.

51. The method of claim 46, wherein Wnt3a is administered hourly, daily, weekly, biweekly or monthly.

52. The method of claim 51, wherein Wnt3a is administered at least once or twice daily.

53. The method of claim 46, wherein the neurological disorder is traumatic brain injury (TBI), stroke, Alzheimer's disease, dementia, Parkinson's Disease, amyotrophic lateral sclerosis (ALS), seizure disorder or epilepsy.

54. The method of claim 46, wherein the neurological disorder is a neurodegenerative disorder or condition.

55. A method of protecting against neuronal cell death comprising administering to a subject having or at risk of having loss of neural function a neuroprotective effective amount of tissue inhibitor of matrix metalloproteinase-3 (TIMP3), wingless-type MMTV integration site family, member 3A (Wnt3a), or combination thereof, thereby protecting against neuronal cell death in the subject.

56. The method of claim 55, wherein the method comprises administering TIMP3.

57. The method of claim 55, wherein the method comprises administering Wnt3a.

58. The method of claim 55, wherein the method comprises administering TIMP3 in combination with Wnt3a.

59. The method of claim 55, wherein the subject is a mammal.

60. The method of claim 59, wherein the subject is human.

61. The method of claim 55, wherein administration is by intravenous delivery.

62. The method of claim 55, wherein TIMP3 or Wnt3a is administered in a dosage of about 0.0001-1000 mg/kg, about 0.01-1000 mg/kg, or about 0.1-100 mg/kg.

63. The method of claim 55, wherein TIMP3 or Wnt3a is administered hourly, daily, weekly, biweekly or monthly.

64. The method of claim 63, wherein TIMP3 or Wnt3a is administered at least once or twice daily.

65. The method of claim 55, wherein the subject has or is at risk of having traumatic brain injury (TBI) or mild TBI, concussive injury, spinal cord injury, stroke, Alzheimer's disease, dementia, Parkinson's Disease, amyotrophic lateral sclerosis (ALS), anxiety, depression, psychiatric injury, seizure disorder or epilepsy.

66. The method of claim 55, wherein the subject has or is at risk of having a neurodegenerative disorder or condition.

67. A method of enhancing neurogenesis comprising administering to a subject in need thereof a neurogenesis effective amount of tissue inhibitor of matrix metalloproteinase-3 (TIMP3), wingless-type MMTV integration site family, member 3A (Wnt3a), or combination thereof, thereby enhancing neurogenesis in the subject.

68. The method of claim 67, wherein the method comprises administering TIMP3.

69. The method of claim 67, wherein the method comprises administering Wnt3a.

70. The method of claim 67, wherein the method comprises administering TIMP3 in combination with Wnt3a.

71. The method of claim 67, wherein the subject is a mammal.

72. The method of claim 71, wherein the subject is human.

73. The method of claim 67, wherein administration is by intravenous delivery.

74. The method of claim 67, wherein TIMP3 or Wnt3a is administered in a dosage of about 0.0001-1000 mg/kg, about 0.01-1000 mg/kg, or about 0.1-100 mg/kg.

75. The method of claim 67, wherein TIMP3 or Wnt3a is administered hourly, daily, weekly, biweekly or monthly.

76. The method of claim 75, wherein TIMP3 or Wnt3a is administered at least once or twice daily.

77. The method of claim 67, wherein the subject has or is at risk of having traumatic brain injury (TBI), stroke, Alzheimer's disease, dementia, Parkinson's Disease, amyotrophic lateral sclerosis (ALS), seizure disorder or epilepsy.

78. The method of claim 67, wherein the subject has or is at risk of having a neurodegenerative disorder or condition.

79. A method of treating a neurological disorder in a subject in need thereof comprising administering an effective amount of tissue inhibitor of matrix metalloproteinase-3 (TIMP3), wingless-type MMTV integration site family, member 3A (Wnt3a), or combination thereof, thereby treating the neurological disorder in the subject.

80. The method of claim 79, wherein the method comprises administering TIMP3.

81. The method of claim 79, wherein the method comprises administering Wnt3a.

82. The method of claim 79, wherein the method comprises administering TIMP3 in combination with Wnt3a.

83. The method of claim 79, wherein the subject is a mammal.

84. The method of claim 83, wherein the subject is human.

85. The method of claim 79, wherein administration is by intravenous delivery.

86. The method of claim 79, wherein TIMP3 or Wnt3a is administered in a dosage of about 0.0001-1000 mg/kg, about 0.01-1000 mg/kg, or about 0.1-100 mg/kg.

87. The method of claim 79, wherein TIMP3 or Wnt3a is administered hourly, daily, weekly, biweekly or monthly.

89. The method of claim 87, wherein TIMP3 or Wnt3a is administered at least once or twice daily.

90. The method of claim 79, wherein the neurological disorder is traumatic brain injury (TBI), stroke, Alzheimer's disease, dementia, Parkinson's Disease, amyotrophic lateral sclerosis (ALS), seizure disorder or epilepsy.

91. The method of claim 79, wherein the neurological disorder is a neurodegenerative disorder or condition.

92. A pharmaceutical composition comprising:
a) at least two neuroprotective polypeptides, the polypeptides comprising tissue inhibitor of matrix metalloproteinase-3 (TIMP3) and wingless-type MMTV integration site family, member 3A (Wnt3a); and
b) a pharmaceutical carrier.

93. A pharmaceutical composition comprising:
a) one or more nucleic acid molecules encoding at least two neuroprotective polypeptides, the polypeptides comprising tissue inhibitor of matrix metalloproteinase-3 (TIMP3) and wingless-type MMTV integration site family, member 3A (Wnt3a); and
b) a pharmaceutical carrier.

94. A method of reducing or inhibiting inflammation of the central nervous system in a subject in need thereof, comprising administering an effective amount of TIMP3, thereby reducing or inhibiting inflammation in central nervous system in the subject.

95. The method of claim 94, wherein the inflammation is brain inflammation.
